(19) United States
(12) Patent Application Publication
(10) Pub. No.: US 2004/0202649 A1
(43) Pub. Date: Oct. 14, 2004

(54) Identification of the MECA-79 antigen and related methods of treating L-selectin-mediated conditions

(75) Inventors: Minoru Fukuda, San Diego, CA (US); Jiunn-Chern Yeh, La Jolla, CA (US); Nobuyoshi Hiraoka, San Diego, CA (US)

Correspondence Address: Cathryn Campbell, McDERMOTT, WILL & EMERY, Suite 700, 4370 La Jolla Village Drive, San Diego, CA, 92122, US

(73) Assignee: The Burnham Institute

(21) Appl. No.: 10/841,707

(22) Filed: May 6, 2004

Related U.S. Application Data
(62) Division of application No. 09/569,320, filed on May 11, 2000.

(51) Int. Cl. A61K 39/395; A61K 31/737

(52) U.S. Cl. 424/130.1; 514/54

(57) ABSTRACT

The present invention provides the structure of the MECA-79 antigen and methods of treating L-selectin-mediated conditions by modulating enzymes that are required for formation of this antigen.

This application was made with government support under CA 71932, CA 48737 and CA 33000 awarded by the National Institutes of Health. The government has certain rights in the invention.

### BACKGROUND OF THE INVENTION

1. Field of the Invention

This invention relates generally to lymphocyte homing and pathologies involving chronic or acute inflammation mediated by L-selectin and, more specifically, to identification of the L-selectin ligand antigen, MECA-79.

2. Background Information

In mammals, lymphocytes circulate in the vascular and lymphatic compartments, allowing maximum exposure of lymphocytes to foreign pathogens. Lymphocytes leave the vascular compartment at lymph nodes, traverse the lymphatic organs, and then return to the vascular system. This directed flow of lymphocytes is dependent on carbohydrate ligands present on specialized endothelial cells, known as high endothelial venules (HEV; Arbones et al., Immunity 1:247-260 (1994)). Although the structure of these carbohydrate ligands is unknown, lymphocyte binding to HEV depends on sialic acid on HEV and can be inhibited by fucosylated sulfated oligosaccharides (Rosen and Bertozzi, Curr. Biol. 261:261-264 (1996)). The homing receptor on lymphocytes is L-selectin, which contains an amino-terminal carbohydrate-binding domain similar to that of the hepatic lectin. Carbohydrate-binding activity of these lectins is calcium-dependent, and they are therefore termed “C-type” lectins (Drickamer, “Molecular Structure of Animal Lectins” in Fukuda and Hindsgaul (Eds), Molecular Glycobioloqy Oxford University Press: Oxford, U.K. (1994)). Counterreceptors (ligands) on HEV capture circulating lymphocytes via L-selectin-dependent adhesion, leading to transmigration. It has been shown that L-selectin is required for this process (Arbones et al., supra, 1994).

The HEV-expressed counterreceptors (ligands) for L-selectin have thus far eluded molecular identification. Consistent with the presence of a C-type lectin domain at the amino terminus of L-selectin, all of the ligands identified to date contain carbohydrate-based recognition determinants. In mouse lymph nodes, two such ligands have been identified as GlyCAM-1 and CD34, both of which are sialomucins (Lasky et al., Cell 69:927-938 (1992); Baumhueter et al., Science 262:436-438 (1993)). CD34 is a type I transmembrane glycoprotein, whereas GlyCAM-1 is a secreted molecule that lacks a transmembrane domain. Additionally, MadCAM-1, which contains a mucin domain in addition to Ig-like domains, can function as a ligand for L-selectin in Peyer's patches (Berg et al., Nature 366:695-698 (1993); and Bargatze et al. , Immunity 3:99-108 (1995)). Four human glycoprotein ligands have been biochemically identified, and two of these have been cloned as CD34 and podocalyxin (Berg et al., J. Cell Biol. 114:343-349 (1991); Puri et al., J. Cell Biol. 131:261-270 (1995); and Sassetti et al., J. Exp. Med. 187:1965-1975 (1998)). All of the human and murine ligands are sialomucin-like, (Puri et al., supra, 1995), and CD34 and podocalyxin have a similar overall domain structure (FIG. 1) with significant sequence homology in their cytoplasmic domains (Sassetti et al., supra, 1998). Notably, only certain glycoforms react with L-selectin. For example, naturally occurring forms of GlyCAM-1, MadCAM-1, CD34 and podocalyxin exist which fail to bind L-selectin due to the absence of necessary post-translational modification (Berg et al., Nature 366:695-698 (1993); Puri et al., supra, 1995; Sassetti et al., supra, 1998; and Dowbenko et al., J. Clin. Invest. 92:952-960 (1993)). Thus, although CD34 and podocalyxin are widely distributed on vascular endothelium, a limited number of vessels (including HEV) express L-selectin-reactive glycoforms (Sassetti et al., supra, 1998; and Baumhueter et al., Blood 84:2554-2565 (1994)).

GlyCAM-1 and CD34 were originally identified as L-selectin ligands in extracts of mouse lymph nodes using a recombinant L-selectin/IgG chimera (Lasky et al., supra, 1992; Baumhueter et al., supra, 1993; and Imai et al., J. Cell Biol. 113:1213-1221 (1991)). Furthermore, a monoclonal antibody, MECA-79, stains HEV in mouse lymph nodes and blocks both lymphocyte attachment to HEV in vitro and short-term homing of lymphocytes to lymph nodes in vivo (Streeter et al., Nature 331:41-43 (1988)). The MECA-79 monoclonal is remarkable in that it reacts with HEV across a wide range of species including mouse and human (Girard et al., FASEB J. 12:603-612 (1998)). Significantly, MECA-79 and L-selectin/IgG stain the same complex of glycoproteins in mouse and human lymphoid organs (Sassetti et al., supra, 1998; and Hemmerich et al., J. Exp. Med. 180:2219-2226 (1994)). This complex of four or more glycoproteins defined by reactivity with MECA-79 is known as peripheral lymph node addressin (PNAd). Although the structure of the MECA-79 antigen has eluded identification, the epitope is believed to be sulfated (Hemmerich et al., supra, 1994) and, in particular, to include a GlcNAc-6-sulfate modification (Kimura et al., Proc. Natl. Acad. Sci. 96:4530-4535 (1999)). Furthermore, previous characterization indicates that the MECA-79 epitope is independent of sialylation and fucosylation (Hemmerich et al., supra, 1994; and Maly et al., Cell 86:643-653 (1996). Nevertheless, the physiologically relevant sulfated structures necessary for L-selectin ligand activity remain to be identified.

L-selectin and its ligands are implicated in lymphocyte recruitment in a variety of chronic inflammatory diseases, and L-selectin ligand activity including MECA-79 expression is induced on microvascular venular endothelium in rheumatoid arthritis, lymphocytic thyroiditis, and inflammatory bowel diseases such as Crohn's disease and ulcerative colitis (Michie et al., Am. J. Pathol. 143:1688-1698 (1993); and Salmi et al., Gastroenterology 106:596-605 (1994)). Increased MECA-79 expression also is associated with nonobese diabetes in the mouse and with transplant rejection (Hanninen et al., J. Clin. Invest. 92:2509-2515 (1993); and Toppila et al., Am. J. Pathol. 155:1303-1310 (1999)).

Methods of controlling L-selectin activity would be desirable in order to reduce inflammatory responses mediated by L-selectin. Such methods could be used to treat or prevent conditions such as acute or chronic inflammation; allograft rejection; or tumor metastasis. However, methods of specifically controlling L-selectin activity await elucidation of the sulfated carbohydrate structure on L-selectin ligands, and identification of the enzymes that manufacture the L-selectin ligand carbohydrate determinants.

Thus, there is a need for identification of the L-selectin ligand carbohydrate structure and identification of the enzyme or enzymes that produce this structure. The present invention satisfies this need and provides related advantages as well.

### SUMMARY OF THE INVENTION

The present invention provides a method of modifying an acceptor molecule by contacting the acceptor molecule with an isolated β1,3GnT, or an active fragment thereof, under conditions that allow addition of core 1 GlcNAc linkages to the acceptor molecule, where the β1,3GnT or active fragment thereof directs expression of a MECA-79 antigen. A β1,3GnT useful for modifying an acceptor molecule according to a method of the invention can have, for example, substantially the amino acid sequence of human β1,3GnT (SEQ ID NO: 2) or substantially the amino acid sequence of murine β1,3GnT (SEQ ID NO: 4).

The invention also provides a method of treating or preventing an L-selectin-mediated condition in a subject by reducing the expression or activity of a β1,3GnT that directs expression of a MECA-79 antigen. In a method of the invention, the expression or activity of a β1,3GnT can be reduced, for example, by administering to a subject an oligosaccharide L-selectin antagonist that inhibits the binding of L-selectin to a MECA-79 antigen. Such an L-selectin antagonist can contain, for example, the oligosaccharide Galβ1→4(SO₃→6) GlcNAcβ1→3Galβ1→3GalNAc or the oligosaccharide NeuNAcα2→3Galβ1→4[sulfo→6(Fucα1→3)GlcNAc]β1→3Galβ1→3GalNAcα1, or, in another embodiment, multimers of one or both of these oligosaccharides. In a further embodiment, an L-selectin-mediated condition is treated or prevented by administering to the subject inhibitory antibody material that specifically binds β1,3GnT. In yet a further embodiment, an L-selectin-mediated condition is treated or prevented by administering to the subject a β1,3GnT antisense nucleic acid molecule that has, for example, at least 20 nucleotides complementary to SEQ ID NO: 1 or SEQ ID NO: 3. In another embodiment, a method of the invention is practiced by reducing the expression or activity of a β1,3GnT that directs expression of a MECA-79 antigen in combination with reducing the expression or activity of L-selectin sulfotransferase-2 (LSST-2) in the subject.

The present invention also provides an isolated L-selectin antagonist containing an extended core 1 structure which includes the oligosaccharide Galβ1→4(SO₃→6)GlcNAcβ1→3Galβ1→3GalNAc. In a further embodiment, the invention provides an isolated L-selectin antagonist containing the oligosaccharide NeuNAcα2→3Galβ1→4[sulfo→6(Fucα1→3)GlcNAc]β1→3Galβ1→3GalNA cα1. In yet another embodiment, an isolated L-selectin antagonist of the invention contains multimers of one or both the the oligosaccharides

Galβ1→4(SO₃→6)GlcNAcβ1→3Galβ1→3GalNAc or

Galβ1→4(SO₃→6)GlcNAcβ1→3Galβ1→3GalNAc.

The present invention also provides an isolated polypeptide which contains an amino acid sequence encoding a L-selectin sulfotransferase-2 (LSST-2), or an active fragment thereof, that directs expression of a MECA-79 antigen in Chinese hamster ovary (CHO) cells. An isolated polypeptide of the invention can have, for example, substantially the amino acid sequence of human LSST-2 (SEQ ID NO: 6).

The present invention further provides substantially purified antibody material that specifically binds a LSST-2 that directs expression of a MECA-79 antigen in CHO cells. Such antibody material, which can be polyclonal or monoclonal antibody material, specifically binds, for example, human LSST-2 having the amino acid sequence SEQ ID NO: 6.

The present invention further provides an isolated nucleic acid molecule which contains a nucleic acid sequence encoding a LSST-2 or an active fragment thereof that directs expression of a MECA-79 antigen in CHO cells. An isolated nucleic acid molecule of the invention can encode, for example, a LSST-2 that has substantially the amino acid sequence of human LSST-2 (SEQ ID NO: 6) and can be, for example, SEQ ID NO: 5. The invention further provides vectors and related host cells that contain a nucleic acid molecule encoding a LSST-2 or active fragment thereof that directs expression of a MECA-79 antigen in CHO cells. In one embodiment, such a vector is a mammalian expression vector.

The invention also provides an isolated antisense nucleic acid molecule which contains a nucleotide sequence that specifically binds to SEQ ID NO: 5, shown in FIG. 4. Such an isolated antisense nucleic acid molecule can have, for example, at least 20 nucleotides complementary to SEQ ID NO: 5. In one embodiment, an isolated antisense nucleic acid molecule contains a nucleotide sequence complementary to the sequence ATG.

Also provided herein is an oligonucleotide, which contains a nucleotide sequence having at least 10 contiguous nucleotides of SEQ ID NO: 5, or a nucleotide sequence complementary thereto. An oligonucleotide of the invention can have, for example, at least 15 contiguous nucleotides of SEQ ID NO: 5, or a nucleotide sequence complementary thereto.

The present invention also provides a method of modifying an acceptor molecule by contacting the acceptor molecule with an isolated LSST-2, or an active fragment thereof, under conditions that allow addition of a sulfate to a GlcNAc acceptor molecule, where the LSST-2 or active fragment thereof directs expression of a MECA-79 antigen in CHO cells. A LSST-2 useful for modifying an acceptor molecule according to a method of the invention can have, for example, substantially the amino acid sequence of human LSST-2 (SEQ ID NO: 6) or an active fragment thereof.

The invention also provides a method of treating or preventing an L-selectin-mediated condition in a subject by reducing the expression or activity of a LSST-2 that directs expression of a MECA-79 antigen in CHO cells. In one embodiment, an L-selectin-mediated condition is treated or prevented by administering to the subject inhibitory antibody material that specifically binds LSST-2. In another embodiment, an L-selectin-mediated condition is treated or prevented by administering to the subject a LSST-2 antisense nucleic acid molecule that has, for example, at least 20 nucleotides complementary to SEQ ID NO: 5.

The invention also provides an isolated polypeptide that contains an amino acid sequence encoding substantially the amino acid sequence of intestinal GlcNAc 6-sulfotransferase (I-GlcNAc6ST) or an active fragment thereof. Such a polypeptide of the invention can have, for example, substantially the amino acid sequence of SEQ ID NO: 8.

In addition, the invention also provides substantially purified antibody material that specifically binds an isolated polypeptide having an amino acid sequence encoding substantially the amino acid sequence of I-GlcNAc6ST or an active fragment thereof. Such antibody material, which can be polyclonal or monoclonal antibody material, specifically binds, for example, I-GlcNAc6ST having the amino acid sequence SEQ ID NO: 8.

The present invention further provides an isolated nucleic acid molecule which contains a nucleic acid sequence encoding an I-GlcNAc6ST or an active fragment thereof. An isolated nucleic acid molecule of the invention can encode, for example, an I-GlcNAc6ST having substantially the amino acid sequence of murine I-GlcNAc6ST (SEQ ID NO: 8) and can be, for example, SEQ ID NO: 7. The invention further provides vectors and related host cells that contain a nucleic acid molecule encoding an I-GlcNAc6ST or active fragment thereof. In one embodiment, such a vector is a mammalian expression vector.

The invention also provides an isolated antisense nucleic acid molecule which contains a nucleotide sequence that specifically binds to SEQ ID NO: 7, shown in FIG. 9. Such an isolated antisense nucleic acid molecule can have, for example, at least 20 nucleotides complementary to SEQ ID NO: 7. In one embodiment, an isolated antisense nucleic acid molecule contains a nucleotide sequence complementary to the sequence ATG.

Also provided herein is an oligonucleotide, which contains a nucleotide sequence having at least 10 contiguous nucleotides of SEQ ID NO: 7, or a nucleotide sequence complementary thereto. An oligonucleotide of the invention can have, for example, at least 15 contiguous nucleotides of SEQ ID NO: 7, or a nucleotide sequence complementary thereto.

The present invention also provides a method of modifying an acceptor molecule by contacting the acceptor molecule with an isolated I-GlcNAc6ST, or an active fragment thereof, under conditions that allow addition of a sulfate to a GlcNAc acceptor molecule.

### BRIEF DESCRIPTION OF THE DRAWINGS

FIG. 1 shows a model of lymph node HEV ligands for L-selectin. Four sialomucins recognized by MECA-79 are shown. GlyCAM-1, CD34, and Sgp200 have been identified in mouse lymph node. CD34, podocalyxin and Sgp200 have been identified in human tonsils. The complex, defined by purification with MECA-79, is denoted the peripheral lymph node addressin (PNAd). The cDNA encoding Sgp200 (sulfated glycoprotein of 200 kd) has yet to be cloned. White circles designate posttranslational modifications including sialylation, fucosylation, and sulfation. CD34 and podocalyxin share the same overall structural organization, each having an amino-terminal mucin domain, a presumed globular domain with cysteines, a transmembrane domain, and homologous cytoplasmic tails.

FIG. 2 shows the human β1,3GnT nucleotide sequence (SEQ ID NO: 1) and predicted amino acid sequence (SEQ ID NO: 2).

FIG. 3 shows the murine β1,3GnT nucleotide sequence (SEQ ID NO: 3) and predicted amino acid sequence (SEQ ID NO: 4).

FIG. 4 shows the human L-selectin sulfotransferase-2 (hLSST-2) nucleotide sequence (SEQ ID NO: 5) and predicted amino acid sequence (SEQ ID NO: 6).

FIG. 5 shows a CLUSTALW alignment of mouse β3GalT-I, -II, -III and -IV and mouse β3GnT proteins. Conserved residues are shaded. White arrows mark the positions of the cysteine residues conserved among β3GalT proteins. The black arrow shows the position of the cysteines conserved in the five proteins.

FIG. 6 shows in vitro substrate specificity of human β1,3GnT.

FIG. 7 shows MECA-79 staining of transfected CHO/CD34 cells.

FIG. 8 shows the results of a rolling experiment performed with four stably transfected CHO cell lines. Open circles represent the CHO/CD34/FT7/hLSST-2 cell line. Open squares represent the CHO/CD34/FT7/hLSST-2/C2GnT-L cell line. Filled squares represent the CHO/CD34/FT7/hLSST-2/core 1 extension β1,3GnT cell line. Filled circles represent the CHO/CD34/FT7/hLSST-2/C2GnT-L/core 1 extension β1,3GnT line cell.

FIG. 9 shows the murine intestinal-GlcNAc 6-sulfotransferase (I-GlcNAc6ST) nucleotide sequence (SEQ ID NO: 7) and predicted amino acid sequence (SEQ ID NO: 8).

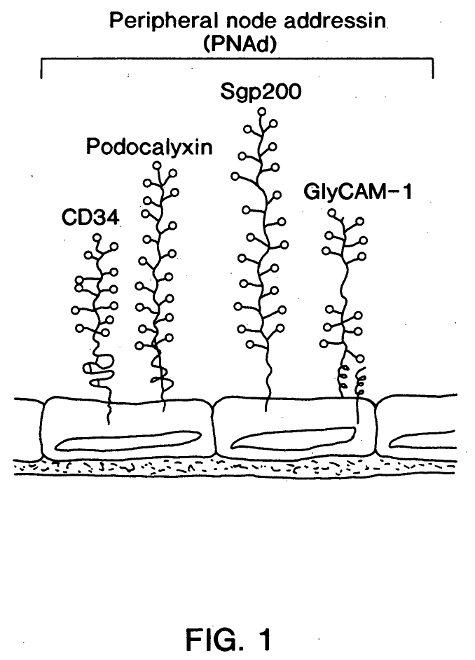

### DETAILED DESCRIPTION OF THE INVENTION

Lymphocyte homing is important for the surveillance of foreign pathogens. Extravasation of lymphocytes in peripheral lymph nodes is mediated through L-selectin binding to L-selectin ligands, sulfated sialyl Lewis^{x} present on high endothelial venules (HEV) Recently cloned L-selectin ligand sulfotransferases (LSST or HEC-GlcNac6ST) form core 2-based selectin ligand functional in rolling assays (Hiraoka et al., Immunity 11:79-89 (1999), and Bistrup et al., J. Cell. Biol. 145:899-910 (1999)). The expression of LSST is highly restricted to HEV, while the sulfotransferase GlcNAc6ST is more widely present and less specific in acceptor substrate requirement.

Analysis of core 2 GnT-leukocyte type knockout mice has indicated that lymphocyte homing and expression of MECA-79 antigen persist even after the gene for the leukocyte type core 2 GnT has been inactivated (Ellies et al., Immunity 9:881-890 (1998)). Structural analysis of L-selectin ligands in HEV of the knockout mice demonstrated that the major oligosaccharides remaining are based on extended core 1 structure such as NeuNAcα2→3Galβ1→4[sulfo→6(Fucα1→3)GlcNAc]β1→3Galβ1→3GalNA cα1→R. As disclosed herein, a novel β1,3-N-acetylglucosaminyl-transferase has been isolated that extends core 1 and forms GlcNAβ1→3Galβ1→3GalNAcα1→R. As further disclosed herein, human L-selectin sulfotransferase-2 (hLSST-2), is unique in the ability to produce, when co-transfected into CHO cells together with β1,3-GnT, NeuNAcα2→3Galβ1→4[sulfo→6(Fucα1→3)GlcNAc]β1→3Galβ1→3GalNAcα1→R, resulting in expression of the MECA-79 epitope. As further disclosed herein, oligosaccharides produced in CHO cells expressing both human β1,3GnT and human LSST-2 support L-selectin-mediated lymphocyte rolling (see Example III). These results demonstrate that 6-sulfo sialyl Lewis X structures on core 1 or core 2 oligosaccharides can serve as L-selectin ligands on high endothelial venules.

Thus, the present invention is directed to the long-awaited discovery of the structure of the L-selectin ligand, MECA-79, and to identification of a β1,3-N-acetylglucosaminyl transferase (β1→3GnT) and a human sulfotransferase (hLSST-2) that can produce this ligand when co-expressed in CHO cells. These discoveries provide a basis for diagnosing and treating L-selectin-mediated conditions, including acute and chronic inflammation, transplant rejection and tumor metastasis.

The present invention relates to an isolated polypeptide which contains an amino acid sequence encoding a β1,3GnT, or an active fragment thereof, that directs expression of a MECA-79 antigen in CHO cells. Such an isolated polypeptide can have, for example, substantially the amino acid sequence of human β1,3GnT (SEQ ID NO: 2) or substantially the amino acid sequence of murine β1,3GnT (SEQ ID NO: 4).

The term “β1,3-N-acetylglucosaminyl transferase,” as used herein, is synonymous with “β1,3GnT” and means an enzyme that catalyzes the β1→3 linkage of a N-acetylglucosamine (GlcNAc) residue to an acceptor molecule. A β1,3GnT useful in the invention is a core 1 extension enzyme and, therefore, catalyzes the β1→3 linkage of a GlcNAc residue to the core 1 structure Galβ1→3GalNAc→R.

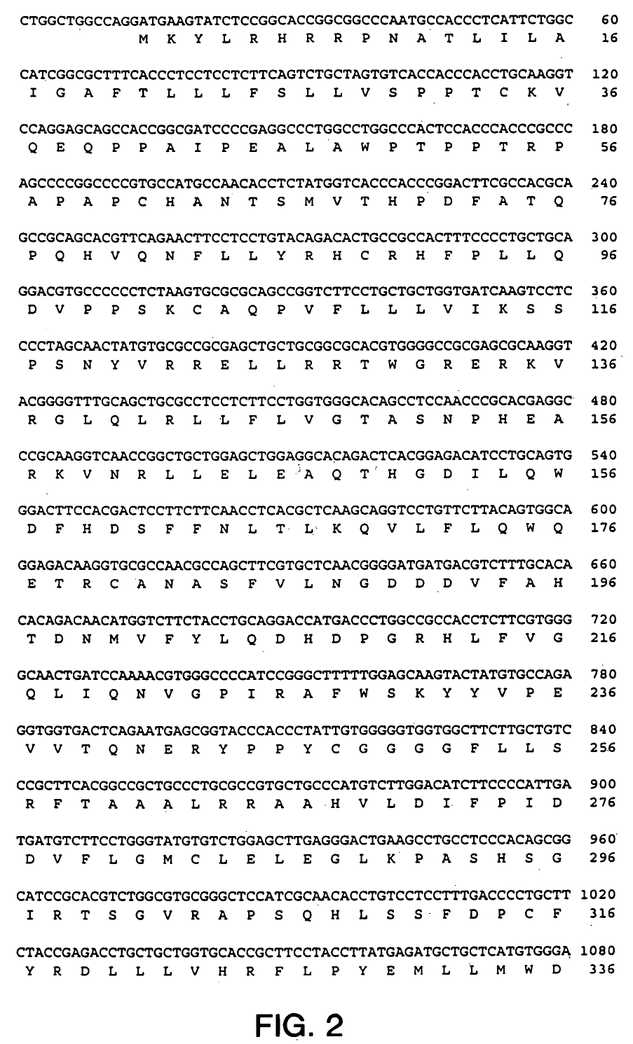

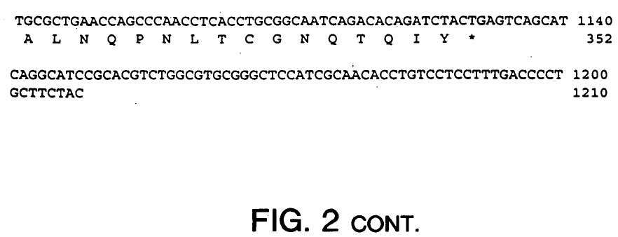

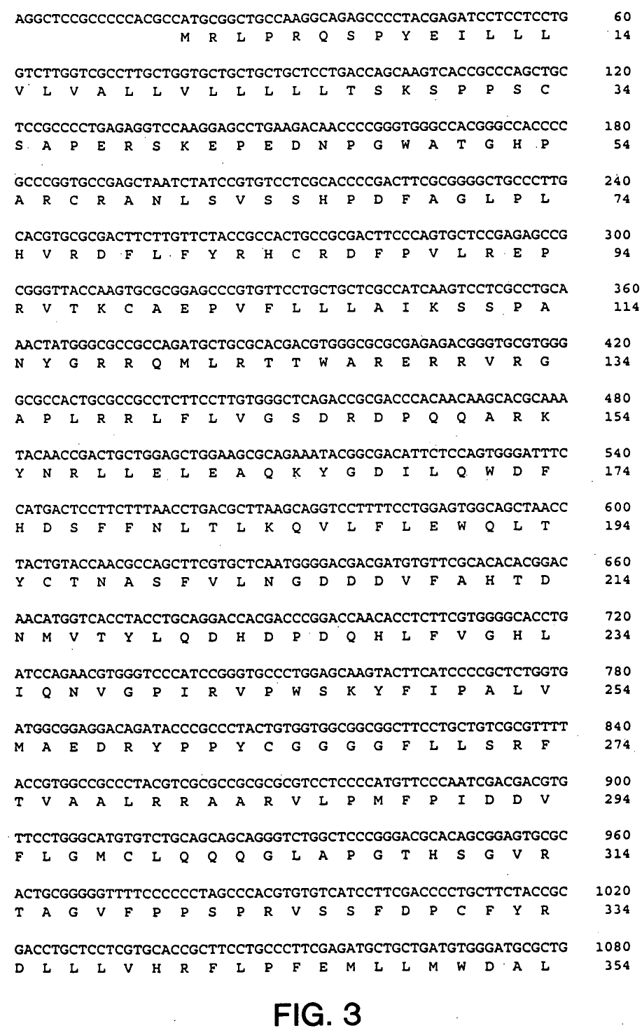

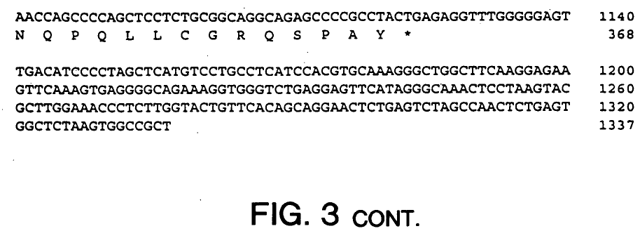

A β1,3GnT that directs expression of a MECA-79 epitope can have, for example, substantially the amino acid sequence of the human β1,3GnT shown in FIG. 2 as SEQ ID NO: 2 or substantially the amino acid sequence of the murine β1,3GnT shown in FIG. 3 as SEQ ID NO: 4. Human β1,3GnT polypeptide (SEQ ID NO: 2) is a type II membrane protein of 352 amino acids. Human β1,3GnT (SEQ ID NO: 2) shares 66.5% amino acid identity with murine β1,3GnT (SEQ ID NO: 4). Regions highly conserved between human and murine β1,3GnT are present, for example, at amino acids 158 to 245, 263 to 322 and 330 to 361 of SEQ ID NO: 2. As disclosed in Example IB, human β1,3GnT (SEQ ID NO: 2) forms the MECA-79 antigen when expressed with L-selectin ligand sulfotransferase-2 in Chinese hamster ovary (CHO) cells. Thus, such a β1,3 GnT is characterized, in part, by the ability to direct expression of a MECA-79 antigen.

The mouse monoclonal antibody, MECA-79, stains HEV in mouse lymph nodes and blocks lymphocyte attachment to HEV in vitro as well as short-term homing of lymphocytes to lymph nodes in vivo (Streeter et al., supra, 1988). Furthermore, the MECA-79 monoclonal antibody reacts with HEV across a variety of species and stains the same complex of glycoproteins in mouse and human lymphoid organs (Girard et al., supra, 1998; Sassetti et al., supra, 1998; Hemmerich et al. supra, 1994). Thus, while the carbohydrate-based recognition determinants on the HEV-expressed L-selectin ligands CD34, podocalyxin, Sgp200 and GlyCAM-2 remain unknown, these L-selectin ligands contain the MECA-79 antigen (Hemmerich, supra, 1994).

As used herein, the term “MECA-79 antigen” means a carbohydrate-containing epitope that specifically reacts with the MECA-79 monoclonal antibody described in Hemmerich, supra, 1994. An exemplary MECA-79 antigen is provided herein as Galβ1→4(SO₃→6)GlcNAcβ1→3Galβ1→3GalNAc. The phrase “directs expression of a MECA-79 antigen” refers to production of a carbohydrate-containing epitope that specifically reacts with the MECA-79 monoclonal antibody. It is understood that an enzyme “directs expression of a MECA-79 antigen” only under the appropriate conditions. Such conditions include availability of a core 1 acceptor molecule and an appropriate donor molecule and further include the presence of one or more additional enzymes. Human β1,3GnT together with the human sulfotransferase LSST-2, but not other sulfotransferases, directs expression of the MECA-79 antigen in CHO cells.

The invention provides a method of treating or preventing an L-selectin-mediated condition in a subject by reducing the expression or activity of a β1,3GnT that directs expression of a MECA-79 antigen. If desired, a method of the invention can be practiced by reducing the expression or activity of a β1,3GnT that directs expression of a MECA-79 antigen in combination with reducing the expression or activity of L-selectin sulfotransferase-2 (LSST-2) in the subject.

As used herein, the term “L-selectin-mediated condition” means any pathology or disorder involving the L-selectin ligand, MECA-79. Such an L-selectin-mediated condition generally can be, for example, acute or chronic inflammation, allograft rejection, or tumor metastasis. An L-selectin-mediated condition also can be, for example, organ transplant rejection, which is typically accompanied by an influx of lymphocytes into the graft. For example, in a rat model of acute cardiac allograft rejection, Toppila et al. demonstrated the induction of L-selectin ligands including MECA-7 on flat-walled venules and capillaries within rejecting cardiac allograft (Toppila et al., Am. J. Pathol. 155:1303-1310 (1999)). Toppila et al. further observed a correlation between the staining intensity of L-selectin ligands on vessels and the severity of acute rejection of heart allografts in humans. L-selectin-mediated conditions further can include rheumatoid arthritis; inflammatory bowel diseases such as Crohn's disease and ulcerative colitis; inflammatory disorders of the skin such as allergic contact dermatitis, psoriasis and Lichen planus; lymphomas; chronic pneumonia; delayed-type hypersensitivity reactions; diabetes; and hyperplastic thymus, each of which are characterized by expression of MECA-79 in HEV-like vessels (Rosen, Am. J. Pathol. 155:1013-1020 (1999); see, also, Table 1). It is understood that these and other conditions of acute or chronic inflammation, allograft rejection or tumor metastasis can be an “L-selectin-mediated” condition that can be treated according to a method of the invention.

TABLE 1
L-selectin-mediated conditions

| Organ | Disease process | Reference |
|---|---|---|
| Synovium | Rheumatoid arthritis | Michie et al., Am. J. Path. |
| | | 143: 1688-1698 (1993); Van |
| | | Dinther-Jansses et al., J. |
| | | Rheum. 17-11-17 (1990) |
| Gut | Crohn's disease | Salmi et al.., |
| | | Gastroenterology 106: 596-605 |
| | | (1994); Duijvestijn et al., |
| | | J. Immunol. 138: 713-719 |
| | | (1987) |
| Gut | Ulcerative colitis | Salmi et al., Eur. J. |
| | | Immunol. 22: 835-843 (1992) |
| Skin | Cutanteous sites of | Michie et al., supra, 1993; |
| | inflammation such as | Arvilommi et al., Eur. J. |
| | allergic contact | Immunol. 26: 825-833 (1996) |
| | dermatitis, psoriasis and | |
| | lichen planus | |
| Skin | Cutaneous lymphomas | Michie et al., supra, 1993 |
| Lung | Chronic interstitial | |
| | pneumonia | |
| Skin | Delayed-type | Mackay et al., Eur. J. |
| | hypersensitivity reaction | Immunol. 22: 835-843 (1992) |
| Pancreas | Diabetes | Hanninen et al., J. Clin. |
| | | Invest. 92: 2509-2515 (1993) |
| Thymus | Hyperplastic thymus | Michie et al., Am. J. Path. |
| | | 147: 412-421 (1995) |

The term “reducing the expression or activity” as used herein to a β1,3GnT, means that the amount of functional β1,3GnT polypeptide or activity is diminished in the subject in comparison with the amount of functional β1,3GnT polypeptide in an untreated subject. Similarly, when used in reference to LSST-2 expression or activity, the term “reduced” means that the amount of functional LSST-2 polypeptide or activity is reduced in the treated subject as compared to an untreated subject. Thus, the term “reduced,” as used herein, encompasses the absence of a β1,3GnT that directs expression of a MECA-79 antigen or a LSST-2, as well as protein expression that is present but reduced as compared to the level of β1,3GnT or LSST-2 expression in an untreated subject. Furthermore, the term reduced refers to suppressed refers to β1,3GnT or LSST-2 protein expression that is diminished throughout the entire domain of β1,3GnT or LSST-2 expression, or to expression that is reduced in some part of the β1,3GnT or LSST-2 expression domain, provided that expression of the MECA-79 antigen is decreased.

As used herein, the term “reduced” also encompasses an amount of β1,3GnT or LSST-2 polypeptide that is equivalent to wild type β1,3GnT or LSST-2 expression, but where the β1,3GnT or LSST-2 polypeptide has a reduced level of activity. For example, mutations within the catalytic domain of β1,3GnT or LSST-2 that reduce glucosaminyltransferase activity or sulfotransferase activity, respectively, are encompassed within the meaning of the term “reduced.”

The present invention relates, in part, to the use of carbohydrate-based drugs for treatment of an L-selectin-mediated condition such as rheumatoid arthritis, inflammatory bowel disease or diabetes. Carbohydrate drugs are well known in the art and include, for example, Acarbose, a maltotetrose analog for treatment of diabetes, which acts as a competitive inhibitor of sucrase and α-amylase (Bayer A G; Balfour and McTavish, Drugs 46:1025 (1993). Other carbohydrate drugs include Relenza™ (GG-167, zanamivir), a sialic acid analog for treatment of influenza which is a selective inhibitor of viral neuramidases (Glaxo Wellcome/Biota; Hayden et al., JAMA 275:295 (1996), and SYNSORB Pk™, an oligosaccharide conjugate for treatment of E. coli 0157. H7 infection developed by SYNSORB Biotech. Additional carbohydrate-based drugs are well known in the art (see, for example, Dumitrui (Ed.), Polysaccharides in Medicinal Applications Dekker, N.Y. (1996)).

In one embodiment, the invention provides a method of treating or preventing an L-selectin-mediated condition in a subject by administering to the subject an oligosaccharide L-selectin antagonist that inhibits the binding of L-selectin to a MECA-79 antigen. Such an L-selectin antagonist can contain, for example, the oligosaccharide Galβ→4(SO₃→6)GlcNAcβ1→3Galβ1→3GalNAc or the oligosaccharide NeuNAcα2→3Galβ1→4[sulfo→6(Fucα1→3) GlcNAc]β1→3Galβ1→3GalNAcα1 or, in another embodiment, multimers of one or both of these oligosaccharides.

As disclosed herein, the MECA-79 epitope has the structure Galβ1→4(SO₃→6)GlcNAcβ1→3Galβ1→3GalNAc and is based on a core 1 structure. As further disclosed herein, an L-selectin ligand contains the MECA-79 related structure NeuNAcα2→3Galβ1→4[sulfo→6(Fucα1→3)GlcNAc]β1→3Galβ1→3GalNA cα1. The term “core 1,” as used herein, means the core structure Galβ1→3GalNAc→R. In conformance with accepted carbohydrate and chemical nomenclature, “Gal” means galactose; “GalNAc” means N-acetylgalactosamine; “GlcNAc” means N-acetylglucosamine; “SO₃” means sulfate; and “NeuNAc” means N-acetylneuraminate, also known as sialic acid. “R” can be a serine or threonine residue of a peptide or protein or, for example, an octyl, O-methyl, p-nitrophenol, amino pyridine, or other convenient moiety.

The term “oligosaccharide,” as used herein, means a linear or branched carbohydrate that consists of from 2 to about 50 monosaccharide units joined by means of glycosidic bonds. The monosaccharide units of an oligosaccharide are polyhydroxy alcohols containing either an aldehyde or a ketone group. An oligosaccharide can have, for example, up to 5, 10, 20, 30, 40 or 50 monosaccharide units. It is understood that “an oligosaccharide L-selectin antagonist” may have other non-carbohydrate components in addition to its carbohydrate component.

An L-selectin antagonist also can be a glycoconjugate or glycomimetic based on the structure Galβ1→4(SO₃→6)GlcNAcβ1→3Galβ1→3GalNAc or NeuNAcα2→3Galβ1→4[sulfo→6(Fucα1→3)GlcNAc]β1→3Galβ1→3GalNAcα1. Thus, an L-selectin antagonist of the invention can be a synthetic glycoconjugate or glycomimetic that retains the ability to inhibit binding of L-selectin to a MECA-79 antigen (Yarema and Bertozzi, Curr. Opin. Chem. Biol. 2:49-61 (1998); Dumitrui, supra, 1996). Multivalent glycoconjugates are particularly useful L-selectin antagonists of the invention.

As disclosed herein, the MECA-79 epitope is formed, in part, by a core 1 extension enzyme (β1,3GnT) which catalyzes the β1→3 linkage of a GlcNAc residue to the core 1 structure (Galβ1→3GalNAc→R) and has the structure Galβ1→4(SO₃→6)GlcNAcβ1→3Galβ1→3GalNAc. Based on this discovery, the present invention provides an oligosaccharide L-selectin antagonist containing an extended core 1 structure which includes the oligosaccharide Galβ1→4(SO₃→6)GlcNAcβ1→3Galβ1→3GalNAc. In one embodiment, an isolated L-selectin antagonist contains the oligosaccharide NeuNAcα2→3Galβ1→4[sulfo→6(Fucα1→3)GlcNAc]β1→3Galβ1→3GalNAcα1. In another embodiment, an L-selectin antagonist contains multimers of one or both of the oligosaccharides Galβ1→4(SO₃→6)GlcNAcβ1→3Galβ1→3GalNAc and NeuNAcα2→3Galβ1→4[sulfo→6(Fucα1→3)GlcNAc]β1→3Galβ1→3GalNA cα1. In addition to the structural features set forth above, an L-selectin antagonist inhibits L-selectin activity, for example, by competing for binding to physiological L-selectin ligand. L-selectin antagonists also include variants of these structures which cannot accept a GlcNAc residue at the 3 position of galactose, such as structures in which C-3 of galactose is deoxy; or variants in which GlcNAc contains a 6-dehydro group. Other L-selectin antagonists can be core 1 structure derivatives which cannot accept a GlcNAc residue at the 3 position of galactose.

In a further embodiment, an L-selectin-mediated condition is treated or prevented by administering to the subject inhibitory antibody material that specifically binds β1,3GnT. In yet a further embodiment, an L-selectin-mediated condition is treated or prevented by administering to the subject a β1,3GnT antisense nucleic acid molecule that has, for example, at least 20 nucleotides complementary to SEQ ID NO: 1 or SEQ ID NO: 3.

The present invention also provides an isolated polypeptide which contains an amino acid sequence encoding a L-selectin sulfotransferase-2 (LSST-2), or an active fragment thereof, that directs expression of a MECA-79 antigen in Chinese hamster ovary (CHO) cells. An isolated polypeptide of the invention can have, for example, substantially the amino acid sequence of human LSST-2 (SEQ ID NO: 6).

As used herein, the term “isolated” means a polypeptide or nucleic acid molecule that is in a form that is relatively free from contaminating lipids, polypeptides, nucleic acids or other cellular material normally associated with the nucleic acid molecule or polypeptide in a cell.

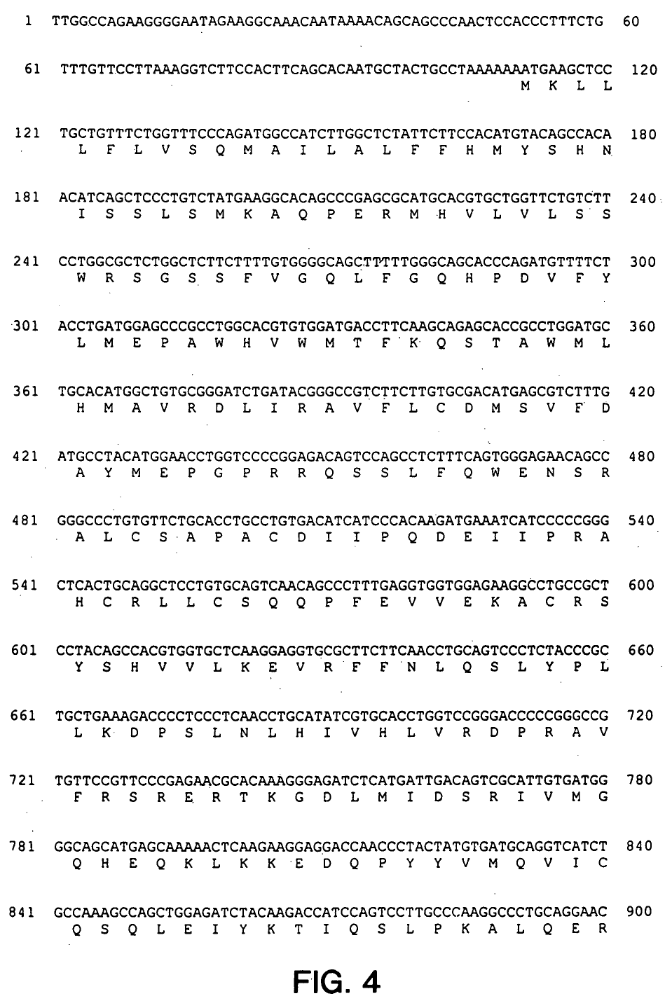

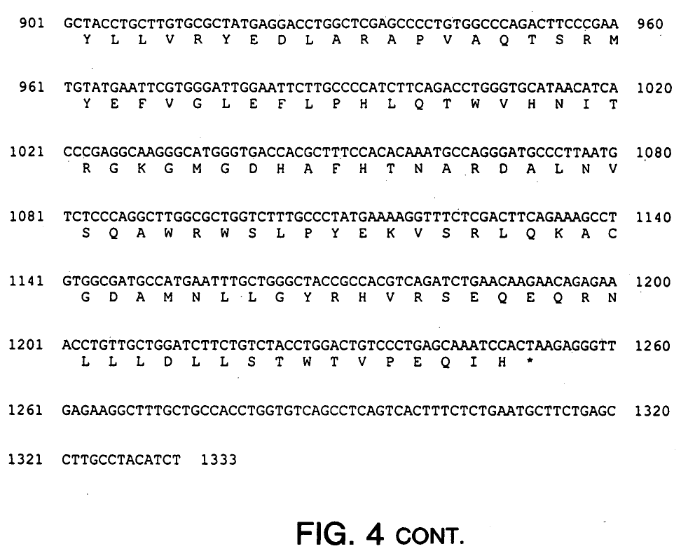

A LSST-2 polypeptide can have substantially the amino acid sequence of SEQ ID NO: 6. Thus, an LSST-2 polypeptide of the invention can be the naturally occurring human LSST-2 (SEQ ID NO: 6), or a related polypeptide having substantial amino acid sequence similarity to this sequence. Such a related polypeptide typically exhibits greater sequence similarity to human LSST-2 than to other sulfotransferases such as murine LSST, and includes isotype variants, alternatively spliced forms and species homologs of the amino acid sequence shown in FIG. 4. As used herein, the term “LSST-2” generally describes polypeptides having an amino acid sequence with greater than about 50% identity, preferably greater than about 60% identity, more preferably greater than about 70% identity, and can be a polypeptide having greater than about 80%, 90%, 95%, 97%, or 99% amino acid sequence identity with SEQ ID NO: 6, said amino acid identity determined with CLUSTALW using the BLOSUM 62 matrix with default parameters, provided that such a polypeptide is able to produce the MECA-79 antigen when expressed in CHO cells under the appropriate conditions. The previously described murine polypeptide, LSST (Hiraoka et al., supra, 1999), which is not able to form the MECA-79 antigen when co-transfected into CHO cells with hβ1,3GnT, therefore is not a LSST-2 polypeptide of the invention.

The present invention also provides active fragments of a LSST-2 polypeptide. As used herein, the term “active fragment” means a polypeptide fragment having substantially the amino acid sequence of a portion of a LSST-2 that directs expression of a MECA-79 antigen in CHO cells, provided that the fragment retains the sulfotransferase activity of the parent polypeptide as well as the ability to direct expression of a MECA-79 antigen in CHO cells. An active fragment of LSST-2 can have, for example, substantially the amino acid sequence of a portion of human LSST-2 (SEQ ID NO:6). Sulfotransferase activity can be assayed, for example, as described in Hiraoka et al., Immunity 11:79-89 (1999). Activity in directing expression of a MECA-79 antigen can be assayed as set forth in Example IB.

As used herein, the term “substantially the amino acid sequence,” when used in reference to a LSST-2 polypeptide or an active fragment thereof, is intended to mean a sequence as shown in FIG. 4, or a similar, non-identical sequence that is considered by those skilled in the art to be a functionally equivalent amino acid sequence. For example, an amino acid sequence that has substantially the amino acid sequence of a human LSST-2 polypeptide (SEQ ID NO: 6) can have one or more modifications such as amino acid additions, deletions or substitutions relative to the amino acid sequence of SEQ ID NO: 6, provided that the modified polypeptide retains substantially the ability to direct expression of a MECA-79 antigen in CHO cells, as described further below.

Thus, it is understood that limited modifications can be made to a human LSST-2 polypeptide or another polypeptide of the invention (see below), or to an active fragment thereof without destroying its biological function. A modification can be, for example, an addition, deletion, or substitution of one or more conservative or non-conservative amino acid residues; substitution of a compound that mimics amino acid structure or function; or addition of chemical moieties such as amino or acetyl groups. The activity of a modified LSST-2 polypeptide or fragment thereof can be assayed by transfecting an encoding nucleic acid molecule into CHO cells and assaying for expression of MECA-79 as disclosed herein.

A particularly useful modification of a polypeptide of the invention, or fragment thereof, is a modification that confers, for example, increased stability. Incorporation of one or more D-amino acids is a modification useful in increasing stability of a polypeptide or polypeptide fragment. Similarly, deletion or substitution of lysine can increase stability by protecting against degradation.

The present invention also provides substantially purified antibody material that specifically binds a LSST-2 that directs expression of a MECA-79 antigen in CHO cells. Such antibody material, which can be polyclonal or monoclonal antibody material, specifically binds, for example, human LSST-2 having the amino acid sequence SEQ ID NO: 6.

A LSST-2 polypeptide or polypeptide fragment can be useful to prepare substantially purified antibody material that specifically binds a LSST-2 which directs expression of a MECA-79 antigen in CHO cells. Such antibody material can be, for example, substantially purified polyclonal antiserum or monoclonal antibody material. The antibody material of the invention be useful, for example, in determining the level of LSST-2 polypeptide in a subject.

As used herein, the term “antibody material” is used in its broadest sense to include polyclonal and monoclonal antibodies, as well as polypeptide fragments of antibodies that retain a specific binding activity for a LSST-2 polypeptide of at least about 1×10⁵ M⁻¹. One skilled in the art would know that anti-LSST-2 antibody fragments such as Fab, F(ab′)² and Fv fragments can retain specific binding activity for a LSST-2 polypeptide and, thus, are included within the definition of antibody material. In addition, the term “antibody material,” as used herein, encompasses non-naturally occurring antibodies and fragments containing, at a minimum, one V_{H} and one V_{L} domain, such as chimeric antibodies, humanized antibodies and single chain Fv fragments (scfv) that specifically bind a LSST-2 polypeptide. Such non-naturally occurring antibodies can be constructed using solid phase peptide synthesis, produced recombinantly or obtained, for example, by screening combinatorial libraries consisting of variable heavy chains and variable light chains as described by Borrebaeck (Ed.), Antibody Engineering (Second edition) New York: Oxford University Press (1995)).

Antibody material “specific for” a LSST-2 polypeptide, or that “specifically binds” a LSST-2 polypeptide, binds with substantially higher affinity to that polypeptide than to an unrelated polypeptide. The substantially purified antibody material of the invention also can bind with significantly higher affinity to a LSST-2 that directs expression of a MECA-79 antigen in CHO cells than to another sulfotransferase that does not direct expression of a MECA-79 antigen in CHO cells.

Anti-LSST-2 antibody material can be prepared, for example, using a LSST-2 fusion protein or a synthetic peptide encoding a portion of a LSST-2 polypeptide such as SEQ ID NO: 6 as an immunogen. One skilled in the art would know that purified LSST-2 polypeptide, which can be produced recombinantly, or fragments of LSST-2, including peptide portions of LSST-2 such as synthetic peptides, can be used as an immunogen. Non-immunogenic fragments or synthetic peptides of LSST-2 can be made immunogenic by coupling the hapten to a carrier molecule such as bovine serum albumin (BSA) or keyhole limpet hemocyanin (KLH). In addition, various other carrier molecules and methods for coupling a hapten to a carrier molecule are well known in the art are described, for example, by Harlow and Lane, Antibodies: A Laboratory Manual (Cold Spring Harbor Laboratory Press, 1988)).

The term “substantially purified,” as used herein in reference to antibody material, means that the antibody material is substantially devoid of polypeptides, nucleic acids and other cellular material which with an antibody is normally associated in a cell. The claimed antibody material that specifically binds an LSST-2 further is substantially devoid of antibody material of unrelated specificities, i.e. that does not specifically bind a LSST-2. The antibody material of the invention can be prepared in substantially purified form, for example, by LSST-2 affinity purification of polyclonal anti-LSST-2 antisera, by screening phage displayed antibodies against a LSST-2 polypeptide such as SEQ ID NO: 6, or as monoclonal antibodies prepared from hybridomas.

The present invention further provides an isolated nucleic acid molecule which contains a nucleic acid sequence encoding a LSST-2 or an active fragment thereof that directs expression of a MECA-79 antigen in CHO cells. An isolated nucleic acid molecule of the invention can encode, for example, a LSST-2 that has substantially the amino acid sequence of human LSST-2 (SEQ ID NO: 6) and can be, for example, SEQ ID NO: 5. The invention further provides vectors and related host cells that contain a nucleic acid molecule encoding a LSST-2 or active fragment thereof that directs expression of a MECA-79 antigen in CHO cells. In one embodiment, such a vector is a mammalian expression vector.

The term “nucleic acid molecule” is used broadly to mean any polymer of two or more nucleotides, which are linked by a covalent bond such as a phosphodiester bond, a thioester bond, or any of various other bonds known in the art as useful and effective for linking nucleotides. Such nucleic acid molecules can be linear, circular or supercoiled, and can be single stranded or double stranded DNA or RNA or can be a DNA/RNA hybrid.

A sense or antisense nucleic acid molecule or oligonucleotide of the invention also can contain one or more nucleic acid analogs. Nucleoside analogs or phosphothioate bonds that link the nucleotides and protect against degradation by nucleases are particularly useful in a nucleic acid molecule or oligonucleotide of the invention. A ribonucleotide containing a 2-methyl group, instead of the normal hydroxyl group, bonded to the 2′-carbon atom of ribose residues, is an example of a non-naturally occurring RNA molecule that is resistant to enzymatic and chemical degradation. Other examples of non-naturally occurring organic molecules include RNA containing 2′-aminopyrimidines, such RNA being 1000× more stable in human serum as compared to naturally occurring RNA (see Lin et al., Nucl. Acids Res. 22:5229-5234 (1994); and Jellinek et al., Biochemistry 34:11363-11372 (1995)).

Additional nucleotide analogs also are well known in the art. For example, RNA molecules containing 2′-0-methylpurine substitutions on the ribose residues and short phosphorothioate caps at the 3′- and 5′-ends exhibit enhanced resistance to nucleases (Green et al., Chem. Biol. 2:683-695 (1995). Similarly, RNA containing 2′-amino-2′-deoxypyrimidines or 2′-fluro-2′-deoxypyrimidines is less susceptible to nuclease activity (Pagratis et al., Nature Biotechnol. 15:68-73 (1997). Furthermore, L-RNA, which is a stereoisomer of naturally occurring D-RNA, is resistant to nuclease activity (Nolte et al., Nature Biotechnol. 14:1116-1119 (1996); Klobmann et al., Nature Biotechnol. 14:1112-1115 (1996). Such RNA molecules and methods of producing them are well known and routine (see Eaton and Piekern, Ann. Rev. Biochem. 64:837-863 (1995). DNA molecules containing phosphorothioate linked oligodeoxynucleotides are nuclease resistant (Reed et al., Cancer Res. 50:6565-6570 (1990). Phosphorothioate-3′hydroxypropylamine modification of the phosphodiester bond also reduces the susceptibility of a DNA molecule to nuclease degradation (see Tam et al., Nucl. Acids Res. 22:977-986 (1994), which is incorporated herein by reference). Furthermore, thymidine can be replaced with 5-(1-pentynyl)-2′-deoxoridine (Latham et al., Nucl. Acids Res. 22:2817-2822 (1994). It is understood that nucleic acid molecules, including antisense molecules and oligonucleotides, containing one or more nucleotide analogs are encompassed by the invention.

The invention also provides vectors containing a nucleic acid molecule encoding a LSST-2. Such vectors can be cloning vectors or expression vectors and provide a means to transfer an exogenous nucleic acid molecule into a host cell, which can be a prokaryotic or eukaryotic cell. Contemplated vectors include those derived from a virus, such as a bacteriophage, a baculovirus or a retrovirus, and vectors derived from bacteria or a combination of bacterial and viral sequences, such as a cosmid or a plasmid. The vectors of the invention can advantageously be used to clone or express LSST-2 or an active fragment thereof. Various vectors and methods for introducing such vectors into a host cell are described, for example, in Ausubel et al., Current Protocols in Molecular Biolosy John Wiley & Sons, Inc. New York (1999).

In addition to a nucleic acid molecule encoding a LSST-2 or active fragment thereof, a vector of the invention also can contain, if desired, one or more of the following elements: an oligonucleotide encoding, for example, a termination codon or a transcription or translation regulatory element; one or more selectable marker genes, such as an ampicillin, tetracycline, neomycin, hygromycin or zeomycin resistance gene, which is useful for selecting stable transfectants in mammalian cells; one or more enhancer or promoter sequences, which can be obtained, for example, from a viral, bacterial or mammalian gene; transcription termination and RNA processing signals, which are obtained from a gene or a virus such as SV40; an origin of replication such as an SV40, polyoma or E. coli origin of replication; versatile multiple cloning sites; and one or more RNA promoters such as a T7 or SP6 promoter, which allows for in vitro transcription of sense and antisense RNA.

In one embodiment, a vector of the invention is an expression vector. Expression vectors are well known in the art and provide a means to transfer and express an exogenous nucleic acid molecule in a host cell. Contemplated expression vectors include vectors that provide for expression in a host cell such as a bacterial cell, yeast cell, insect cell, frog cell, mammalian cell or other animal cell. Such expression vectors include regulatory elements specifically required for expression of the DNA in a cell, the elements being located relative to the nucleic acid molecule encoding LSST-2 so as to permit expression thereof. The regulatory elements can be chosen to provide constitutive expression or, if desired, inducible or cell type-specific expression. Regulatory elements required for expression have been described above and include transcription and translation start sites and termination sites. Such sites permit binding, for example, of RNA polymerase and ribosome subunits. A bacterial expression vector can include, for example, an RNA transcription promoter such as the lac promoter, a Shine-Delgarno sequence and an initiator AUG codon in the proper frame to allow translation of an amino acid sequence.

Mammalian expression vectors can be particularly useful and can include, for example, a heterologous or homologous RNA transcription promoter for RNA polymerase binding, a polyadenylation signal located downstream of the coding sequence, an AUG start codon in the appropriate frame and a termination codon to direct detachment of a ribosome following translation of the transcribed mRNA. Commerciallv available mammalian expression vectors include pSI, which contains the SV40 enhancer/promoter (Promega; Madison, Wis.); pTarget™ and pCI, which each contain the cytomegalovirus (CMV) enhancer/promoter (Promega); pcDNA3.1, a CMV expression vector (Invitrogen; Carlsbad, Calif.); and pRc/RSV, which contains Rous sarcoma virus (RSV) enhancer/promoter sequences (Invitrogen). In addition to these constitutive mammalian expression vectors, inducible expression systems are available, including, for example, an ecdysone-inducible mammalian expression system such as pIND and pVgRXR from Invitrogen. These and other mammalian expression vectors are commercially available or can be assembled by those skilled in the art using well known methods. An example of a eukaryotic expression vector of the invention is pcDNA1.1/LSST-2, described in Example II below.

The invention also provides a host cell containing a vector that includes a nucleic acid molecule encoding a LSST-2 or an active fragment thereof. Such a host cell can be used to replicate the vector and, if desired, to express and isolate substantially pure recombinant LSST-2 using well known biochemical procedures (see Ausubel, supra, 1999). In addition, a host cell of the invention can be used in an in vitro or in vivo method to transfer sulfate to an acceptor molecule. Such host cells can be chosen or transfected to additionally co-express one or more additional enzymes involved in oligosaccharide biosynthesis, for example, the core 1 extension enzyme, hβ1,3GnT. Such host cells can be used to prepare ligands having high affinity for the L-selectin glycoprotein receptor.

Host cells expressing LSST-2 or an active fragment thereof also can be used to screen for selective inhibitors of LSST-2 or for agents that selectively react with a L-selectin ligand. These agents can be administered to a subject to prevent or treat an L-selectin-mediated condition as described further below.

Examples of host cells useful in the invention include bacterial, yeast, frog and mammalian cells. Various mammalian cells useful as host cells include, for example, mouse NIH/3T3 cells, CHO cells, COS cells and HeLa cells. In addition, mammalian cells obtained, for example, from a primary explant culture are useful as host cells. Additional host cells include non-human mammalian embryonic stem cells, fertilized eggs and embryos, which can be routinely used to generate transgenic animals, such as mice, which express the novel LSST-2 of the invention. Transgenic mice expressing LSST-2 can be used, for example, to screen for compounds that enhance or inhibit the MECA-79 producing activity of this enzyme. Methods for introducing a vector into a host including electroporation, microinjection, calcium phosphate, DEAE-dextran and lipofection methods well known in the art (see, for example, Ausubel, supra, 1999).

The invention also provides an isolated antisense nucleic acid molecule which contains a nucleotide sequence that specifically binds to SEQ ID NO: 5, shown in FIG. 4. Such an isolated antisense nucleic acid molecule can have, for example, at least 20 nucleotides complementary to SEQ ID NO: 5. In one embodiment, an isolated antisense nucleic acid molecule contains a nucleotide sequence complementary to the sequence ATG.

An isolated antisense nucleic acid molecule can be useful to reduce LSST-2 expression, thereby treating or preventing an L-selectin-mediated condition in a subject. Antisense nucleic acid molecules can, for example, reduce mRNA translation or increase mRNA degradation and thereby suppress gene expression (see, for example, Galderisi et al., J. Cell Physiol. 181:251-257 (1999)). Methods of using antisense nucleic acid molecules as therapeutic agents are well known in the art (see Galderisi et al., supra, 1999; Alama et al., Pharmacol. Res. 36:171-178 (1997); and Temsamani et al., Biotechnol. Appl. Biochem. 26 (part 2):65-71 (1997))

The skilled artisan will recognize that effective reduction of LSST-2 expression depends upon the antisense nucleic acid molecule having a high percentage of homology with the endogenous LSST-2 locus, for example, the endogenous human locus SEQ ID NO: 5. A nucleic acid molecule encoding human LSST-2 (SEQ ID NO: 5) provided herein is useful in the antisense methods of the invention.

The homology requirement for effective suppression of gene expression using antisense methodology can be determined empirically. In general, a minimum of about 80-90% nucleic acid sequence identity is preferred for effective suppression of LSST-2 expression. More preferably, a nucleic acid molecule that is exactly homologous to the gene to be suppressed is used as an antisense nucleic acid molecule. Both antisense oligonucleotides of 20, 22, 25, 30, 35, 40 or more nucleotides, as well as antisense nucleic acid molecules is expressed in a vector are contemplated for use in the antisense methods of the invention.

Also provided herein is an oligonucleotide, which contains a nucleotide sequence having at least 10 contiguous nucleotides of SEQ ID NO: 5, or a nucleotide sequence complementary thereto. An oligonucleotide of the invention can have, for example, at least 15 contiguous nucleotides of SEQ ID NO: 5, or a nucleotide sequence complementary thereto.

Oligonucleotides of the invention can advantageously be used, for example, as primers for PCR or sequencing, as probes for research or diagnostic applications, and in therapeutic applications. An oligonucleotide of the invention can incorporate, if desired, a detectable moiety such as a radiolabel, fluorochrome, luminescent tag, ferromagnetic substance, or a detectable agent such as biotin, and used to detect expression of LSST-2 in a cell or tissue. Those skilled in the art can determine the appropriate length and nucleic acid sequence of a LSST-2 oligonucleotide for a particular application. An oligonucleotide of the invention contains a nucleotide sequence having, for example, at least, 10, 12, 14, 16, 18, 20, 25, 30, 35 or 40 contiguous nucleotides of SEQ ID NO: 5, or a nucleotide sequence complementary thereto.

The present invention also provides a method of modifying an acceptor molecule by contacting the acceptor molecule with an isolated LSST-2, or an active fragment thereof, under conditions that allow addition of a sulfate to a GlcNAc acceptor molecule, where the LSST-2 or active fragment thereof directs expression of a MECA-79 antigen in CHO cells. A LSST-2 useful for modifying an acceptor molecule according to a method of the invention can have, for example, substantially the amino acid sequence of human LSST-2 (SEQ ID NO: 6) or an active fragment thereof. In a method of the invention, an isolated LSST-2 can add a sulfate to the 6-position of GlcNAc.

The term “acceptor molecule,” as used herein, refers to a molecule that is acted upon, or “modified,” by a protein-having enzymatic activity. For example, an acceptor molecule can be a molecule that accepts the transfer of a sulfate due to the sulfotransferase activity of a LSST-2 polypeptide. An acceptor molecule can be in substantially pure form or in an impure form such as in a host cell or cellular extract. An acceptor molecule can be a naturally occurring molecule or a completely or partially synthesized molecule. An acceptor molecule can contain one or more sugar residues prior to modification and can be further modified to contain additional sugar residues. An acceptor molecule useful in the invention contains the core 1 structure (Galβ1→3GalNAc→R) and can be, for example, CD34 as disclosed herein. Additional acceptor molecules include podocalyxin, Sgp200 and GlyCAM-1.

In one embodiment, the invention provides a method of modifying an acceptor molecule by contacting the acceptor molecule with an isolated LSST-2 or an active fragment thereof in combination with an isolated β1,3GnT that directs expression of a MECA-79 antigen under conditions that allow addition of core 1 GlcNAc linkages and sulfate to the acceptor molecule such that a MECA-79 antigen is formed. As disclosed herein, human β1,3GnT (SEQ ID NO: 2) and human LSST-2 (SEQ ID NO: 6) can be used together to modify a core 1 structure to produce the MECA-79 antigen, Galβ1→4(SO₃→6)GlcNAcβ1→3Galβ1→3GalNAc, in CHO cells.

The invention also provides a method of treating or preventing an L-selectin-mediated condition in a subject by reducing the expression or activity of a LSST-2 that directs expression of a MECA-79 antigen in CHO cells. L-selectin-mediated conditions as well as techniques for reducing the expression or activity of an enzyme such as LSST-2 are described hereinabove.

As further disclosed herein in Example IV, the mouse intestinal GlcNAc 6-sulfotransferase can, in combination with a β1,3GnT, for the MECA-79 antigen in Lec2 cells, but not in CHO cells. In these cells, which are defective in Golgi sialylation, more core 1 extension product is formed by the core 1 extension enzyme, β1,3GnT. Under these conditions, murine intestinal GlcNAc 6-sulfotransferase (I-GlcNAc6ST) adds enough sulfate to form the MECA-79 antigen. Thus, the invention also provides a novel nucleic acid molecule that contains a nucleic acid sequence encoding substantially the amino acid sequence of I-GlcNAc6ST or an active fragment thereof. An isolated nucleic acid molecule of the invention can encode, for example, substantially the amino acid sequence of SEQ ID NO: 8 and can be, for example, SEQ ID NO: 7. In one embodiment, an isolated nucleic acid molecule of the invention encodes substantially the amino acid sequence of SEQ ID NO: 8, provided that the nucleic acid molecule is not AI115260.

The invention also provides an isolated polypeptide that contains an amino acid sequence encoding substantially the amino acid sequence of intestinal GlcNAc 6-sulfotransferase (I-GlcNAc6ST) or an active fragment thereof. Such a polypeptide of the invention can have, for example, substantially the amino acid sequence of SEQ ID NO: 8.

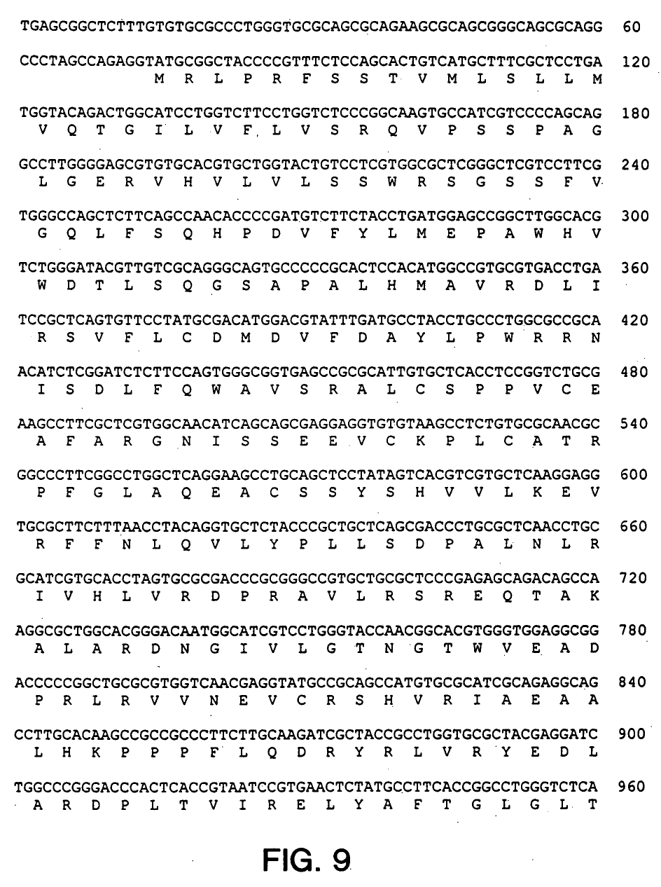

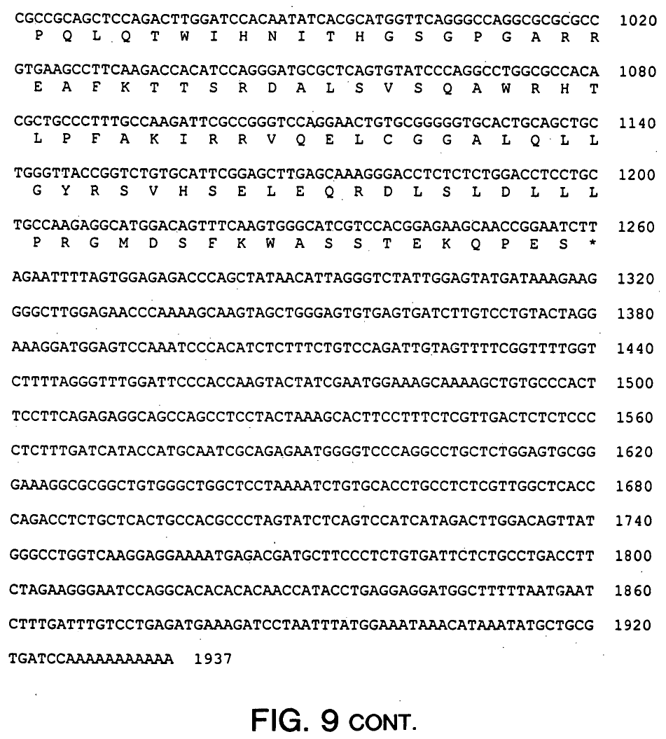

An I-GlcNAc6ST polypeptide has substantially the amino acid sequence of SEQ ID NO: 8. Thus, an I-GlcNAc6ST polypeptide of the invention can be the naturally occurring I-GlcNAc6ST (SEQ ID NO: 8), or a related polypeptide having substantial amino acid sequence similarity to this sequence. Such a related polypeptide includes isotype variants, alternatively spliced forms and species homologs of the amino acid sequence shown in FIG. 9. As used herein, the term “I-GlcNAc6ST” generally describes polypeptides having an amino acid sequence with greater than about 50% identity, preferably greater than about 60% identity, more preferably greater than about 70% identity, and can be a polypeptide having greater than about 75%, 80%, 85%, 90%, 95%, 97%, or 99% amino acid sequence identity with SEQ ID NO: 8, said amino acid identity determined with CLUSTALW using the BLOSUM 62 matrix with default parameters, provided that such a polypeptide is able to produce the MECA-79 antigen when expressed in Lec2 cells under the appropriate conditions. The previously described murine polypeptide, LSST (Hiraoka et al., supra, 1999) is not an I-GlcNAc6ST polypeptide of the invention.

The present invention also provides active fragments of an I-GlcNAc6ST polypeptide. As used herein, The term “active fragment,” when used in reference to an I-GlcNAc6ST polypeptide, means a polypeptide fragment having substantially the amino acid sequence of a portion of an I-GlcNAc6ST, provided that the fragment retains the 6-sulfotransferase activity of the parent polypeptide as well as the ability to direct expression of a MECA-79 antigen when expressed in Lec2 cells. An active fragment can have, for example, substantially the amino acid sequence of a portion of murine I-GlcNAc6ST (SEQ ID NO:8). Sulfotransferase activity can be assayed, for example, as described in Hiraoka et al., Immunity 11:79-89 (1999). Activity in directing expression of a MECA-79 antigen can be assayed as set forth in Example IB.

Furthermore, the term “substantially the amino acid sequence,” when used in reference to an I-GlcNAc6ST polypeptide or an active fragment thereof, is intended to mean a sequence as shown in FIG. 9, or a similar, non-identical sequence that is considered by those skilled in the art to be a functionally equivalent amino acid sequence. For example, an amino acid sequence that has substantially the amino acid sequence of an I-GlcNAc6ST polypeptide (SEQ ID NO: 8) can have one or more modifications such as amino acid additions, deletions or substitutions relative to the amino acid sequence of SEQ ID NO: 8, provided that the modified polypeptide retains substantially 6-sulfotransferase activity as well as the ability to direct expression of a MECA-79 antigen in Lec2 cells (see Example IV).

In addition, the invention also provides substantially purified antibody material that specifically binds an isolated polypeptide having an amino acid sequence encoding substantially the amino acid sequence of I-GlcNAc6ST or an active fragment thereof. Such antibody material, which can be polyclonal or monoclonal antibody material, specifically binds, for example, murine I-GlcNAc6ST having the amino acid sequence SEQ ID NO: 8. Thus, such antibody material includes polyclonal and monoclonal antibodies, as well as polypeptide fragments of antibodies that retain a specific binding activity for an I-GlcNAc6ST polypeptide of at least about 1×10⁵ M⁻¹. As set forth above, such antibody material includes Fab, F(ab′)₂ and Fv fragments as well as chimeric and humanized antibodies and single chain Fv fragments (scFv) that specifically bind an I-GlcNAc6ST polypeptide of the invention.

The present invention further provides an isolated nucleic acid molecule which contains a nucleic acid sequence encoding an I-GlcNAc6ST or an active fragment thereof. An isolated nucleic acid molecule of the invention can encode, for example, an I-GlcNAc6ST having substantially the amino acid sequence of murine I-GlcNAc6ST (SEQ ID NO: 8) and can be, for example, SEQ ID NO: 7. The invention further provides vectors and related host cells that contain a nucleic acid molecule encoding an I-GlcNAc6ST or active fragment thereof. In one embodiment, the vector is a mammalian expression vector. As set forth above, a variety of vectors, including cloning and expression vectors, and host cells are well known in the art.

The invention also provides an isolated antisense nucleic acid molecule which contains a nucleotide sequence that specifically binds to SEQ ID NO: 7, shown in FIG. 9. Such an isolated antisense nucleic acid molecule can have, for example, at least 20 nucleotides complementary to SEQ ID NO: 7. In one embodiment, an isolated antisense nucleic acid molecule contains a nucleotide sequence complementary to the sequence ATG. An antisense nucleic acid molecule can have, for example, 20, 22, 25, 30, 35, 40 or more nucleotides.

Also provided herein is an oligonucleotide, which contains a nucleotide sequence having at least 10 contiguous nucleotides of SEQ ID NO: 7, or a nucleotide sequence complementary thereto. An oligonucleotide of the invention can have, for example, at least 15 contiguous nucleotides of SEQ ID NO: 7, or a nucleotide sequence complementary thereto.

As set forth above, a sense or antisense nucleic acid molecule or oligonucleotide of the invention is a polymer of two or more nucleotides, which are linked by a covalent bond such as a phosphodiester bond, a thioester bond, or any of various other bonds known in the art as useful and effective for linking nucleotides. Furthermore, a nucleic acid molecule or oligonucleotide of the invention can contain one or more nucleic acid analogs (see above). An oligonucleotide of the invention contains a nucleotide sequence having, for example, at least, 10, 12, 14, 16, 18, 20, 25, 30, 35 or 40 contiguous nucleotides of SEQ ID NO: 7, or a nucleotide sequence complementary thereto.

The present invention also provides a method of modifying an acceptor molecule by contacting the acceptor molecule with an isolated I-GlcNAc6ST, or an active fragment thereof, under conditions that allow addition of a sulfate to a GlcNAc acceptor molecule.

The following examples are intended to illustrate but not limit the present invention.

### EXAMPLE I

### Cloning and Characterization of the Human Core 1 Extension Enzyme, β1,3-N-Acetylglucosaminyltransferase (β1,3GnT)

This example describes the cloning and characterization of human and murine β1,3-N-acetylglucosaminyltransferase (β1,3GnT).

A. Cloning and Characterization of Human β1,3GnT

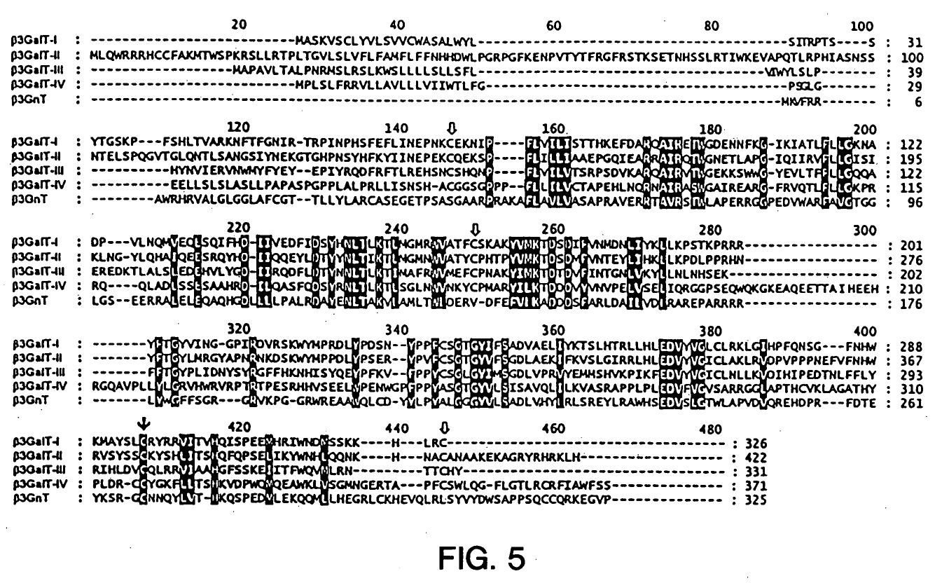

Sequences homologous among β1,3-galactosyl transferases and β1,3-N-acetylglucosaminyltransferases shown in FIG. 5 (Zhou et al., Proc. Natl. Acad. Sci., USA 96:406-411 (1999)) were used as probes to search dbEST using the tblastn program. An EST clone (AB015630) containing a single open reading frame of 372 amino acids was obtained. Primers 5′-CTGGCTGGCCAGGATGAAGTATCTCC-3′ (β1,3GnT-A1; SEQ ID NO: 9) and 5′-CCTGATGCTGACTCAGTAGATCTGTGTC-3′ (β1,3GnT-A2AS; SEQ ID NO: 10) were designed based on EST AB015630. After amplification of single-stranded cDNA prepared from HT29 cells using the Thermoscript RT-PCR system (Gibco-BRL #11146-024; Baithersburg, Md.), a 1.2 kb fragment containing full-length coding sequence was isolated (see FIG. 2). The 1.2 Kb fragment containing the full-length human β1,3GnT cDNA was subcloned into the mammalian expression vector pcDNA3.1 (Invitrogen) and designated pcDNA3.1/hβ1,3GnT-A.

In order to characterize the human β1,3GnT enzyme, a soluble form of the enzyme was prepared by amplifying amino acids 44 to 372 with PCR primers 5′-CGGGATCCCGAGGCCCTGGCCTGGCCCACTCC-3′ (β1,3GnT-A-5′Bam; SEQ ID NO: 11) and 5′-GCTCTAGACTCAGTAGATCTGTGTCTGATTGC-3′ (β1,3GnT-A-3′AS-Xba; SEQ ID NO: 12) and subsequently cloning the amplified fragment into the BamHI and XbaI sites of pcDNA3.1/HSH, a modified vector based on pCDNA3.1/Hydro (Invitrogen) and containing a signal peptide followed by a 6 histidine tag. This vector (4 μg) was transfected into Chinese hamster ovary (CHO) cells using lipofectamine PLUS (Gibco-BRL #10964-013). As a negative control, CHO cells were mock transfected with a vector lacking the β1,3GnT sequence.

Media from cells expressing the soluble enzyme or mock transfected were collected and concentrated essentially as described in Yeh et al., J. Biol. Chem. 274:3215-3221 (1999). For analysis of β1,3-galactosyltransferase activity, ³H-UDP-galactose was used as the sugar nucleotide donor and GalNAc-α-pNP and GlcNAc-β-pNP were used as oligosaccharide acceptor molecules. For detection of β1,3-N-acetylglucosaminyltransferase (β1,3GnT) activity, ³H-UDP-GlcNAc was used as the sugar nucleotide donor with the following oligosaccharide acceptor molecules: Galβ1,3Glc-β-pNP; core 1 pNP (Galβ1,3GalNAc-α-pNP); core 2 pNP (Galβ1,3(GlcNAcβ1,6)GalNAc-α-pNP); Gal-α-pNP and Gal-β-pNP.

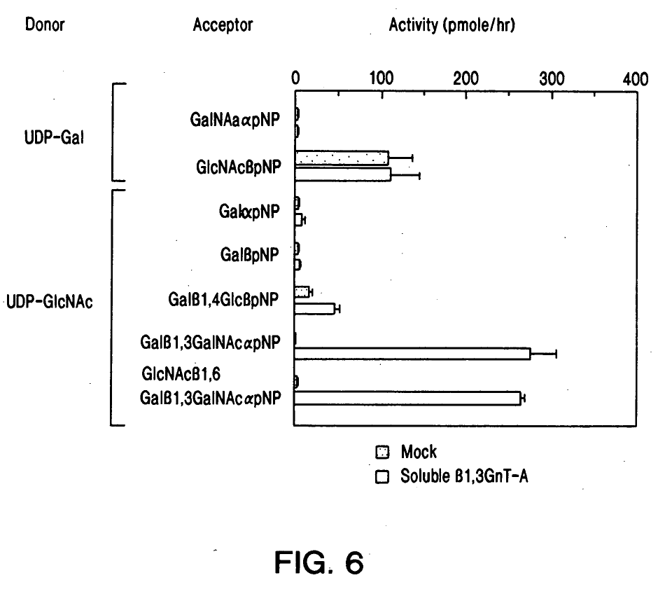

Supernatant from cells expressing the soluble enzyme or mock transfected was assayed for in vitro enzyme activity. As shown in FIG. 6, concentrated medium from soluble enzyme transfected cells was found to have activity in transferring ³H-UDP-GlcNAc to core 1-pNP and core 2-pNP. These results indicate that the cloned enzyme has activity as a core 1 extension β1,3-N-acetylglucosaminyltransferase.

B. Production of the MECA-79 Antigen Using Recombinant hβ1,3GnT (SEQ ID NO: 2)

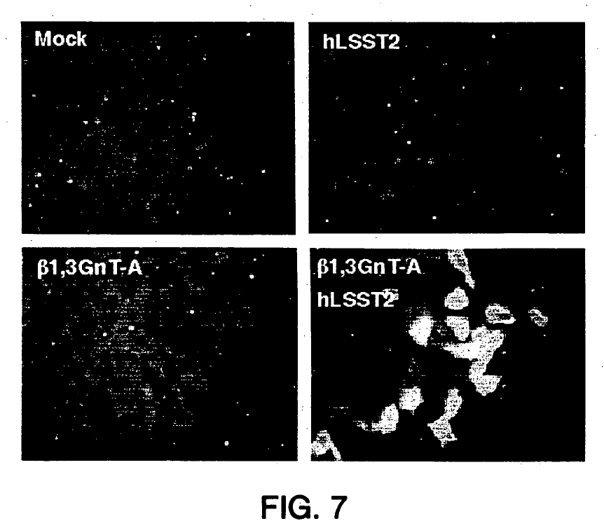

CHO cells were transfected with CD34 and either (a) no enzyme; (b) pcDNA1/hLSST-2 alone; pcDNA3.1/Zeo/mβ1,3GnT alone; or pcDNA1/hLSST-2 and pcDNA3.1/Zeo/mβ1,3GnT together using lipofectamine essentially as described above. Mock transfected and transfected cells were stained with MECA-79 antibody obtained from Pharmingen (San Diego, Calif.), and further incubated with goat anti-rat IgM antibodies essentially as described in Hemmerich et al., supra, 1994. As shown in FIG. 7, positive staining with MECA-79 antibody was only observed in cells co-transfected with both hLSST-2 and mβ1,3GnT vectors, but not in cells only transfected with either enzyme alone. No other sulfotransferases examined showed MECA-79 expression when cotrasnfected into CHO cells with mβ1,3GnT. These results indicate that the human L-selectin sulfotransferase-2 and the core 1 extension enzyme β1,3GnT are sufficient to form the MECA-79 antigen when co-expressed in CHO cells.

C. Cloning and Characterization of Murine β1,3GnT

Several sets of primers based on the human core 1 extension β1,3GnT were used for PCR amplification of single stranded cDNA prepared from mouse small intestine using a SMART PCR cDNA synthesis kit according to the manufacturer's instructions (Clontech #K1052-1). PCR amplification was performed using the following conditions: 94° C. for 2 minutes, followed by 35 cycles of 94° C. for 1 minute, 55° C. for 1 minute, and 72° C. for 1 minute. Only one set of primers gave a specific amplification product. Primers A7 (5′-TTCCTGCTGCTGGTGATCAAGTCC-3′; SEQ ID NO: 13), which corresponds to human β1,3GnT nucleotides 335 to 358) and primer A3AS (5′-CAGGACCTGCTTGAGCGTGAGGTTG-3′; SEQ ID NO: 14), which corresponds to human β1,3GnT nucleotides 560 to 585, gave a product of 251 bp.

5′- and 3′-RACE were performed to isolate additional murine β1,3GnT sequence. 5′-RACE was performed using Marathon-Ready mouse testis cDNA (Clontech) using mA2AS primer 5′-ATGGAAATCCCACTGGAGAATGTCGCCGT-3′ (SEQ ID NO: 15) and the AP1 primer provided by Marathon-Ready cDNA kit. 3′-RACE was performed using mA1 primer 5′-GCCTGCAAACTATGGGCGCCGCCAGAT-3′ (SEQ ID NO: 16) and the SMART primer (Clontech) on mouse small intestine single stranded cDNA prepared using Clontech's SMART PCR cDNA synthesis kit as a template. The full-length cDNA was amplified based on the RACE sequence from mouse small intestine single-stranded cDNA and subcloned into pcDNA3.1/Zeo and designated pcDNA3.1/Zeo/mβ13,GnT.

### EXAMPLE II

### Cloning of Human L-Selectin Ligand Sulfotransferase (LSST-2)

This example describes the isolation of a nucleic acid molecule encoding human L-selectin ligand sulfotransferase-2 (LSST-2), which, together with the β1,3-N-acetylglucosaminyltransferase, directs expression of the MECA-79 antigen.

Like other sulfotransferases in the same gene family (Mazany et al., Biochim. Biophys. Acta 1407:92-97 (1998)), the coding sequence for human LSST-2 was expected to reside in a single exon. Thus, human genomic DNA was used as the template for PCR-based cloning. Primers corresponding to nucleotides 891 to 910 and nucleotides 1327-1302 of mouse LSST-1 (Hiraoka et al., supra, 1999) were used to amplify human genomic DNA as follows. Samples were denatured for 3 minutes at 94° C., followed by 40 cycles of 1 minute at 94° C., 30 seconds at 61° C., and 45 seconds at 72° C. The amplified products were cloned into pBluescript by TA cloning. The resultant coding sequence was 79.2% identical to mouse LSST-1 at the nucleotide level.

To clone the full-length LSST-2 coding sequence, a P1 phage library of human genomic DNA (Genome System Inc.; St. Louis, Mo.) was PCR-amplified using

| | | |
|---|---|---|
| primers | 5′-CCGAATTCTCCCAGAACGCACAAAG-3′ | (SEQ ID |
| | | NO: 17) |
| and | 5′-CCCAAGCTTCTCATAGCGCACAAGCAG-3′. | (SEQ ID |
| | | NO: 18) |

The PCR was carried out for 30 cycles using a 67° C. annealing temperature. From the single positive clone, DNA was purified and sequenced directly. The coding sequence present on the single exon was confirmed by reverse transcriptase (RT)-PCR using poly(A)⁺ RNA isolated from human lymph node, as described previously (Hiraoka et al., supra, 1999). Three pairs of primers used in these PCR reactions correspond to

| | |
|---|---|
| 5′-TTGGCCAGAAGGGGAATAG-3′ | (SEQ ID NO: 19) |
| and | |
| 5′-CCACTGAAAGAGGCTGGACTGT-3′; | (SEQ ID NO: 20) |
| 5′-GGTTCTGTCTTCCTGGCGCTC-3′ | (SEQ ID NO: 21) |
| and | |
| 5′-TTTGGCAGATGACCTGCATCAC-3′; | (SEQ ID NO: 22) |
| and | |
| 5′-AGAACGCACAAAGGAGATCTCA-3′ | (SEQ ID NO: 23) |
| and | |
| 5′-AGATGTAGGCAAGGCTCAGAAG-3′. | (SEQ ID NO: 24) |

PCR with the first two pairs of primers was performed by denaturation for 3 minutes at 94° C., followed by 35 cycles of 1 minute at 94° C., 30 seconds at 56° C., and 1 minute at 72° C. For the PCR with the third pair of primers, the annealing temperature was changed to 55° C. With the first pair of primers (SEQ ID NOS: 19 and 20), the expected characteristic fragment of 470 bp was obtained. With the second pair of primers (SEQ ID NOS: 21 and 22), the expected characteristic fragment of 617 bp was obtained. With the third pair of primers (SEQ ID NOS: 23 and 24), the expected characteristic fragment of 600 bp was obtained.

The cDNA containing full-length coding sequence of human LSST-2 was excised by XbaI and TfiI, blunt-ended and cloned into pcDNA1.1 (Invitrogen). The resulting LSST-2 expression vector, in which the LSST-2 coding sequence is expressed under control of the CMV promoter, was designated pcDNA1.1/LSST-2.

### EXAMPLE III

### Functional Analysis of Human β1,3GnT

This example describes the function of hβ1,3GnT when stably expressed in CHO cells with hLSST-2.

The following CHO cell lines were generated by stable transfection:

CHO/CD34/FT7/hLSST-2;

CHO/CD34/FT7/hLSST-2/C2GnT-L;

CHO/CD34/FT7/hLSST-2/core 1 extension β1,3GnT; and

CHO/CD34/FT7/hLSST-2/C2GnT-L/core 1 extension β1,3GnT

The stable cell lines were established by standard procedures. Cells were selected with a combination of neomycin, hygromycin and zeocin. The expression of each. gene was confirmed by immunostaining with specific antibodies against the relevant cell surface antigens.

Expression of human CD34 was confirmed by the positive staining of cells with anti-human CD34 antibody. CHO/CD34/FT7/hLSST-2 was first established. The expression of human fucosyltransferase 7 (FT7) was confirmed by the positive staining of cells with anti-sialyl Lewis x (product of FT7) antibody 2H5 as described in Kimura et al., Proc. Natl. Acad. Sci., USA 96:4530-4535 (1997). Expression of hLSST-2 was confirmed by transient transfection of β1,3GnT-A (core 1 extension β1,3GnT) and cells were stained with MECA-79 as described above. For the confirmation of C2GnT expression in the CHO/CD34/FT7/hLSST/C2GnT-L cell line, the NCC-ST-439 antibody against sialyl Lewis x core 2 structure was used essentially as described in Kumamoto et al., Biochim. Biophys. Res. Comm. 247:514-517 (1998). For the confirmation of core 1 extension β1,3GnT expression in the CHO/CD34/FT7/hLSST/core 1 extension β1,3GnT cell line, MECA-79.antibody staining was performed as described above.

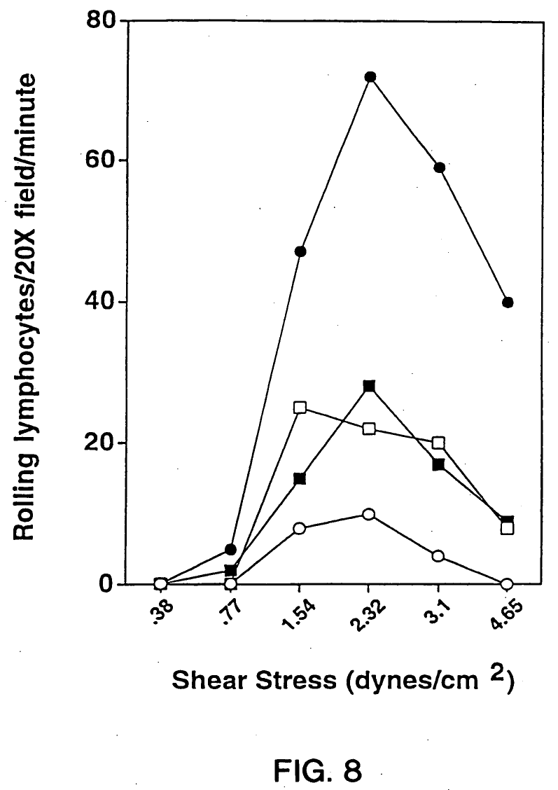

Cells were grown as a monolayer on tissue culture flasks, and mouse lymphocytes were allowed to flow over the monolayer under different shear forces essentially as described in Fuhlbrigge et al., J. Cell Biol. 135:837-48 (1996). The number of lymphocytes which rolled on the cell monolayer were monitored by video camera and counted. As shown in FIG. 8, CHO cells expressing either the core 2 extension enzyme, C2GnT-L (open square) or the human core 1 extension enzyme, β1,3GnT (filled square) rolled more than cells only expressing fucosyltransferase VII (FT7; open circle). Furthermore, rolling was significantly enhanced when lymphocytes rolled on cells expressing both the core 2 extension enzyme, C2GnT-L, and human β1,3GnT (filled circle). These results indicate that both core 1 and core 2 extended sulfo sialyl Lewis X determinants play a role in lymphocyte homing.

### EXAMPLE IV

### Murine Intestinal GlcNAc 6-Sulfotransferase

This example describes the cloning and characterization of the murine intestinal GlcNAc 6-sulfotransferase.

The coding sequence of mouse LSST-1 (Hiraoka et al., Immunity 11:79-89 (1999)) was used as probe to search dbEST using tblstx program. One unknown query. gene (AI115260) was found to have 53.8% identity with the coding regions of mouse LSST-1. A115260 is a sequence isolated from mouse embryo cDNA. Sequence analysis of this cDNA, obtained from Genome Systems (St. Louis, Mo.), revealed that this cDNA encodes a protein of 396 amino acids, designated intestinal GlcNAc 6-sulfotransferase. The cDNA insert was digested with EcoRI and XbaI and cloned into the corresponding sites of pcDNA3.1 (Invitrogen) to produce the expression vector pcDNA3-I-GlcNAc6ST.

Lec2 cells, which are defective in Golgi sialylation due to a CMP-sialic acid transporter defect, were doubly transfected with pcDNA3-I-GlcNAc6ST and pcDNA3.1/hβ1,3GnT-A. Because of the absence of sialic acid in Lec2 cells, core 1 extension occurs with the competition of sialylation and, therefore, more core 1 extended structure is formed by the core 1 extension enzyme β1,3GnT. Under these conditions, the MECA-79 antigen was produced in the doubly transfected Lec2 cells. Similar production of MECA-79 antigen was observed when Lec2 cells were doubly transfected with mLSST-1 and hβ1,3GnT (SEQ ID NO: 2). These results indicate that, under certain conditions, mLSST-1 or I-GLCNAc6ST can form the MECA-79 antigen.

All journal article, reference, and patent citations provided above, in parentheses or otherwise, whether previously stated or not, are incorporated herein by reference.

Although the invention has been described with reference to the examples above, it should be understood that various modifications can be made without departing from the spirit of the invention. Accordingly, the invention is limited only by the following claims.

1. (canceled)

2. A method of treating or preventing an L-selectin-mediated condition in a subject, comprising administering to said subject an oligosaccharide L-selectin antagonist that inhibits the binding of L-selectin to a MECA-79 antigen.

3. The method of claim 2, wherein said L-selectin antagonist comprises the oligosaccharide Galβ1→β4(SO₃→6)GlcNAcβ1→3Galβ1→3GalNAc.

4. The method of claim 3, wherein said L-selectin antagonist comprises NeuNAcα2→3Galβ1→4[sulfo→6(Fucα1→3)GlcNAc]β1→3Galβ1→3GalNAcα1.

5. The method of claim 3, wherein said L-selectin antagonist comprises two or more of the oligosaccharide Galβ1→4(SO₃→6)GlcNAcβ1→3Galβ1→3GalNAc.

6. The method of claim 4, wherein said L-selectin antagonist comprises two or more of the oligosaccharide NeuNAcα2→3Galβ1→4[sulfo→6(Fucα1→3)GlcNAc]β1→3Galβ1→3GalNAcα1.

7-10. (canceled)

11. The method of claim 2, further comprising reducing the expression or activity of L-selectin sulfotransferase-2(LSST-2) in said subject.

12-29. (canceled)


SEQUENCE LISTING (29 sequences)

SEQ ID NO: 1 - DNA, 1208 bp, Homo Sapien
  CDS (14)...(1129)
ctggctggccaggatgaagtatctccggcaccggcggcccaatgccaccctcattctggccatcggcgctttcaccctcctcctcttcagtctgctagtgtcaccacccacctgcaaggtccaggagcagccaccggcgatccccgaggccctggcctggcccactccacccacccgcccagccccggccccgtgccatgccaacacctctatggtcacccacccggacttcgccacgcagccgcagcacgttcagaacttcctcctgtacagacactgccgccactttcccctgctgcaggacgtgcccccctctaagtgcgcgcagccggtcttcctgctgctggtgatcaagtcctcccctagcaactatgtgcgccgcgagctgctgcggcgcacgtggggccgcgagcgcaaggtacggggtttgcagctgcgcctcctcttcctggtgggcacagcctccaacccgcacgaggcccgcaaggtcaaccggctgctggagctggaggcacagactcacggagacatcctgcagtgggacttccacgactccttcttcaacctcacgctcaagcaggtcctgttcttacagtggcaggagacaaggtgcgccaacgccagcttcgtgctcaacggggatgatgacgtctttgcacacacagacaacatggtcttctacctgcaggaccatgaccctggccgccacctcttcgtggggcaactgatccaaaacgtgggccccatccgggctttttggagcaagtactatgtgccagaggtggtgactcagaatgagcggtacccaccctattgtgggggtggtggcttcttgctgtcccgcttcacggccgctgccctgcgccgtgctgcccatgtcttggacatcttccccattgatgatgtcttcctgggtatgtgtctggagcttgagggactgaagcctgcctcccacagcggcatccgcacgtctggcgtgcgggctccatcgcaacacctgtcctcctttgacccctgcttctaccgagacctgctgctggtgcaccgcttcctaccttatgagatgctgctcatgtgggatgcgctgaaccagcccaacctcacctgcggcaatcagacacagatctactgagtcagcatcaggcatccgcacgtctggcgtgcgggctccatcgcaacacctgtcctcctttgacccctgcttctac

SEQ ID NO: 2 - protein, 372 aa, Homo Sapien
MKYLRHRRPNATLILAIGAFTLLLFSLLVSPPTCKVQEQPPAIPEALAWPTPPTRPAPAPCHANTSMVTHPDFATQPQHVQNFLLYRHCRHFPLLQDVPPSKCAQPVFLLLVIKSSPSNYVRRELLRRTWGRERKVRGLQLRLLFLVGTASNPHEARKVNRLLELEAQTHGDILQWDFHDSFFNLTLKQVLFLQWQETRCANASFVLNGDDDVFAHTDNMVFYLQDHDPGRHLFVGQLIQNVGPIRAFWSKYYVPEVVTQNERYPPYCGGGGFLLSRFTAAALRRAAHVLDIFPIDDVFLGMCLELEGLKPASHSGIRTSGVRAPSQHLSSFDPCFYRDLLLVHRFLPYEMLLMWDALNQPNLTCGNQTQIY

SEQ ID NO: 3 - DNA, 1337 bp, Mus musculus
  CDS (19)...(1122)
aggctccgcccccacgccatgcggctgccaaggcagagcccctacgagatcctcctcctggtcttggtcgccttgctggtgctgctgctgctcctgaccagcaagtcaccgcccagctgctccgcccctgagaggtccaaggagcctgaagacaaccccgggtgggccacgggccaccccgcccggtgccgagctaatctatccgtgtcctcgcaccccgacttcgcggggctgcccttgcacgtgcgcgacttcttgttctaccgccactgccgcgacttcccagtgctccgagagccgcgggttaccaagtgcgcggagcccgtgttcctgctgctcgccatcaagtcctcgcctgcaaactatgggcgccgccagatgctgcgcacgacgtgggcgcgcgagagacgggtgcgtggggcgccactgcgccgcctcttccttgtgggctcagaccgcgacccacaacaagcacgcaaatacaaccgactgctggagctggaagcgcagaaatacggcgacattctccagtgggatttccatgactccttctttaacctgacgcttaagcaggtccttttcctggagtggcagctaacctactgtaccaacgccagcttcgtgctcaatggggacgacgatgtgttcgcacacacggacaacatggtcacctacctgcaggaccacgacccggaccaacacctcttcgtggggcacctgatccagaacgtgggtcccatccgggtgccctggagcaagtacttcatccccgctctggtgatggcggaggacagatacccgccctactgtggtggcggcggcttcctgctgtcgcgttttaccgtggccgccctacgtcgcgccgcgcgcgtcctccccatgttcccaatcgacgacgtgttcctgggcatgtgtctgcagcagcagggtctggctcccgggacgcacagcggagtgcgcactgcgggggttttcccccctagcccacgtgtgtcatccttcgacccctgcttctaccgcgacctgctcctcgtgcaccgcttcctgcccttcgagatgctgctgatgtgggatgcgctgaaccagccccagctcctctgcggcaggcagagccccgcctactgagaggtttgggggagttgacatcccctagctcatgtcctgcctcatccacgtgcaaagggctggcttcaaggagaagttcaaagtgaggggcagaaaggtgggtctgaggagttcatagggcaaactcctaagtacgcttggaaaccctcttggtactgttcacagcaggaactctgagtctagccaactctgagtggctctaagtggccgct

SEQ ID NO: 4 - protein, 368 aa, Mus musculus
MRLPRQSPYEILLLVLVALLVLLLLLTSKSPPSCSAPERSKEPEDNPGWATGHPARCRANLSVSSHPDFAGLPLHVRDFLFYRHCRDFPVLREPRVTKCAEPVFLLLAIKSSPANYGRRQMLRTTWARERRVRGAPLRRLFLVGSDRDPQQARKYNRLLELEAQKYGDILQWDFHDSFFNLTLKQVLFLEWQLTYCTNASFVLNGDDDVFAHTDNMVTYLQDHDPDQHLFVGHLIQNVGPIRVPWSKYFIPALVMAEDRYPPYCGGGGFLLSRFTVAALRRAARVLPMFPIDDVFLGMCLQQQGLAPGTHSGVRTAGVFPPSPRVSSFDPCFYRDLLLVHRFLPFEMLLMWDALNQPQLLCGRQSPAY

SEQ ID NO: 5 - DNA, 1333 bp, Homo Sapien
  CDS (111)...(1250)
ttggccagaaggggaatagaaggcaaacaataaaacagcagcccaactccaccctttctgtttgttccttaaaggtcttccacttcagcacaatgctactgcctaaaaaaatgaagctcctgctgtttctggtttcccagatggccatcttggctctattcttccacatgtacagccacaacatcagctccctgtctatgaaggcacagcccgagcgcatgcacgtgctggttctgtcttcctggcgctctggctcttcttttgtggggcagctttttgggcagcacccagatgttttctacctgatggagcccgcctggcacgtgtggatgaccttcaagcagagcaccgcctggatgctgcacatggctgtgcgggatctgatacgggccgtcttcttgtgcgacatgagcgtctttgatgcctacatggaacctggtccccggagacagtccagcctctttcagtgggagaacagccgggccctgtgttctgcacctgcctgtgacatcatcccacaagatgaaatcatcccccgggctcactgcaggctcctgtgcagtcaacagccctttgaggtggtggagaaggcctgccgctcctacagccacgtggtgctcaaggaggtgcgcttcttcaacctgcagtccctctacccgctgctgaaagacccctccctcaacctgcatatcgtgcacctggtccgggacccccgggccgtgttccgttcccgagaacgcacaaagggagatctcatgattgacagtcgcattgtgatggggcagcatgagcaaaaactcaagaaggaggaccaaccctactatgtgatgcaggtcatctgccaaagccagctggagatctacaagaccatccagtccttgcccaaggccctgcaggaacgctacctgcttgtgcgctatgaggacctggctcgagcccctgtggcccagacttcccgaatgtatgaattcgtgggattggaattcttgccccatcttcagacctgggtgcataacatcacccgaggcaagggcatgggtgaccacgctttccacacaaatgccagggatgcccttaatgtctcccaggcttggcgctggtctttgccctatgaaaaggtttctcgacttcagaaagcctgtggcgatgccatgaatttgctgggctaccgccacgtcagatctgaacaagaacagagaaacctgttgctggatcttctgtctacctggactgtccctgagcaaatccactaagagggttgagaaggctttgctgccacctggtgtcagcctcagtcactttctctgaatgcttctgagccttgcctacatct

SEQ ID NO: 6 - protein, 380 aa, Homo Sapien [sequence omitted]

SEQ ID NO: 7 - DNA, 1937 bp, Mus musculus
  CDS (75)...(1259)
tgagcggctctttgtgtgcgccctgggtgcgcagcgcagaagcgcagcgggcagcgcaggccctagccagaggtatgcggctaccccgtttctccagcactgtcatgctttcgctcctgatggtacagactggcatcctggtcttcctggtctcccggcaagtgccatcgtccccagcaggccttggggagcgtgtgcacgtgctggtactgtcctcgtggcgctcgggctcgtccttcgtgggccagctcttcagccaacaccccgatgtcttctacctgatggagccggcttggcacgtctgggatacgttgtcgcagggcagtgcccccgcactccacatggccgtgcgtgacctgatccgctcagtgttcctatgcgacatggacgtatttgatgcctacctgccctggcgccgcaacatctcggatctcttccagtgggcggtgagccgcgcattgtgctcacctccggtctgcgaagccttcgctcgtggcaacatcagcagcgaggaggtgtgtaagcctctgtgcgcaacgcggcccttcggcctggctcaggaagcctgcagctcctatagtcacgtcgtgctcaaggaggtgcgcttctttaacctacaggtgctctacccgctgctcagcgaccctgcgctcaacctgcgcatcgtgcacctagtgcgcgacccgcgggccgtgctgcgctcccgagagcagacagccaaggcgctggcacgggacaatggcatcgtcctgggtaccaacggcacgtgggtggaggcggacccccggctgcgcgtggtcaacgaggtatgccgcagccatgtgcgcatcgcagaggcagccttgcacaagccgccgcccttcttgcaagatcgctaccgcctggtgcgctacgaggatctggcccgggacccactcaccgtaatccgtgaactctatgccttcaccggcctgggtctcacgccgcagctccagacttggatccacaatatcacgcatggttcagggccaggcgcgcgccgtgaagccttcaagaccacatccagggatgcgctcagtgtatcccaggcctggcgccacacgctgccctttgccaagattcgccgggtccaggaactgtgcgggggtgcactgcagctgctgggttaccggtctgtgcattcggagcttgagcaaagggacctctctctggacctcctgctgccaagaggcatggacagtttcaagtgggcatcgtccacggagaagcaaccggaatcttagaattttagtggagagacccagctataacattagggtctattggagtatgataaagaaggggcttggagaacccaaaagcaagtagctgggagtgtgagtgatcttgtcctgtactaggaaaggatggagtccaaatcccacatctctttctgtccagattgtagttttcggttttggtcttttagggtttggattcccaccaagtactatcgaatggaaagcaaaagctgtgcccacttccttcagagaggcagccagcctcctactaaagcacttcctttctcgttgactctctcccctctttgatcataccatgcaatcgcagagaatggggtcccaggcctgctctggagtgcgggaaaggcgcggctgtgggctggctcctaaaatctgtgcacctgcctctcgttggctcacccagacctctgctcactgccacgccctagtatctcagtccatcatagacttggacagttatgggcctggtcaaggaggaaaatgagacgatgcttccctctgtgattctctgcctgaccttctagaagggaatccaggcacacacacaaccatacctgaggaggatggctttttaatgaatctttgatttgtcctgagatgaaagatcctaatttatggaaataaacataaatatgctgcgtgatccaaaaaaaaaaa

SEQ ID NO: 8 - protein, 395 aa, Mus musculus
MRLPRFSSTVMLSLLMVQTGILVFLVSRQVPSSPAGLGERVHVLVLSSWRSGSSFVGQLFSQHPDVFYLMEPAWHVWDTLSQGSAPALHMAVRDLIRSVFLCDMDVFDAYLPWRRNISDLFQWAVSRALCSPPVCEAFARGNISSEEVCKPLCATRPFGLAQEACSSYSHVVLKEVRFFNLQVLYPLLSDPALNLRIVHLVRDPRAVLRSREQTAKALARDNGIVLGTNGTWVEADPRLRVVNEVCRSHVRIAEAALHKPPPFLQDRYRLVRYEDLARDPLTVIRELYAFTGLGLTPQLQTWIHNITHGSGPGARREAFKTTSRDALSVSQAWRHTLPFAKIRRVQELCGGALQLLGYRSVHSELEQRDLSLDLLLPRGMDSFKWASSTEKQPES

SEQ ID NO: 9 - DNA, 26 bp, Homo Sapien
ctggctggccaggatgaagtatctcc

SEQ ID NO: 10 - DNA, 28 bp, Homo Sapien
cctgatgctgactcagtagatctgtgtc

SEQ ID NO: 11 - DNA, 32 bp, Artificial Sequence
  Synthetic Construct
cgggatcccgaggccctggcctggcccactcc

SEQ ID NO: 12 - DNA, 32 bp, Artificial Sequence
  Synthetic Construct
gctctagactcagtagatctgtgtctgattgc

SEQ ID NO: 13 - DNA, 24 bp, Homo sapien
ttcctgctgctggtgatcaagtcc

SEQ ID NO: 14 - DNA, 25 bp, Homo sapien
caggacctgcttgagcgtgaggttg

SEQ ID NO: 15 - DNA, 29 bp, Mus musculus
atggaaatcccactggagaatgtcgccgt

SEQ ID NO: 16 - DNA, 27 bp, Mus musculus
gcctgcaaactatgggcgccgccagat

SEQ ID NO: 17 - DNA, 26 bp, Homo sapien
ccgaattctcccgagaacgcacaaag

SEQ ID NO: 18 - DNA, 27 bp, Homo sapien
cccaagcttctcatagcgcacaagcag

SEQ ID NO: 19 - DNA, 19 bp, Homo sapien
ttggccagaaggggaatag

SEQ ID NO: 20 - DNA, 22 bp, Homo sapien
ccactgaaagaggctggactgt

SEQ ID NO: 21 - DNA, 21 bp, Homo sapien
ggttctgtcttcctggcgctc

SEQ ID NO: 22 - DNA, 22 bp, Homo sapien
tttggcagatgacctgcatcac

SEQ ID NO: 23 - DNA, 22 bp, Homo sapien
agaacgcacaaaggagatctca

SEQ ID NO: 24 - DNA, 22 bp, Homo sapien
agatgtaggcaaggctcagaag

SEQ ID NO: 25 - protein, 326 aa, Mus musculus
MASKVSCLYVLSVVCWASALWYLSITRPTSSYTGSKPFSHLTVARKNFTFGNIRTRPINPHSFEFLINEPNKCEKNIPFLVILISTTHKEFDARQAIRETWGDENNFKGIKIATLFLLGKNADPVLNQMVEQESQIFHDIIVEDFIDSYHNLTLKTLMGMRWVATFCSKAKYVMKTDSDIFVNMDNLIYKLLKPSTKPRRRYFTGYVINGGPIRDVRSKWYMPRDLYPDSNYPPFCSGTGYIFSADVAELIYKTSLHTRLLHLEDVYVGLCLRKLGIHPFQNSGFNHWKMAYSLCRYRRVITVHQISPEEMHRIWNDMSSKKHLRC

SEQ ID NO: 26 - protein, 422 aa, Mus musculus
MLQWRRRHCCFAKMTWSPKRSLLRTPLTGVLSLVFLFAMFLFFNHHDWLPGRPGFKENPVTYTFRGFRSTKSETNHSSLRTIWKEVAPQTLRPHIASNSSNTELSPQGVTGLQNTLSANGSIYNEKGTGHPNSYHFKYIINEPEKCQEKSPFLILLIAAEPGQIEARRAIRQTWGNETLAPGIQIIRVFLLGISIKLNGYLQHAIQEESRQYHDIIQQEYLDTYYNLTIKTLMGMNWVATYCPHTPYVMKTDSDMFVNTEYLIHKLLKPDLPPRHNYFTGYLMRGYAPNRNKDSKWYMPPDLYPSERYPVFCSGTGYVFSGDLAEKIFKVSLGIRRLHLEDVYVGICLAKLRVDPVPPPNEFVFNHWRVSYSSCKYSHLITSHQFQPSELIKYWNHLQQNKHNACANAAKEKAGRYRHRKLH

SEQ ID NO: 27 - protein, 331 aa, Mus musculus
MAPAVLTALPNRMSLRSLKWSLLLLSLLSFLVIWYLSLPHYNVIERVNWMYFYEYEPIYRQDFRFTLREHSNCSHQNPFLVILVTSRPSDVKARQAIRVTWGEKKSWWGYEVLTFFLLGQQAEREDKTLALSLEDEHVLYGDIIRQDFLDTYNNLTLKTIMAFRWVMEFCPNAKYIMKTDTDVFINTGNLVKYLLNLNHSEKFFTGYPLIDNYSYRGFFHKNHISYQEYPFKVFPPYCSGLGYIMSGDLVPRVYEMMSHVKPIKFEDVYVGICLNLLKVDIHIPEDTNLFFLYRIHLDVCQLRRVIAAHGFSSKEIITFWQVMLRNTTCHY

SEQ ID NO: 28 - protein, 371 aa, Mus musculus
MPLSLFRRVLLAVLLLVIIWTLFGPSGLGEELLSLSLASLLPAPASPGPPLALPRLLISNSHACGGSGPPPFLLILVCTAPEHLNQRNAIRASWGAIREARGFRVQTLFLLGKPRRQQLADLSSESAAHRDILQASFQDSYRNLTLKTLSGLNWVNKYCPMARYILKTDDDVYVNVPELVSELIQRGGPSEQWQKGKEAQEETTAIHEEHRGQAVPLLYLGRVHWRVRPTRTPESRHHVSEELWPENWGPFPPYASGTGYVLSISAVQLILKVASRAPPLPLEDVFVGVSARRGGLAPTHCVKLAGATHYPLDRCCYGKFLLTSHKVDPWQMQEAWKLVSGMNGERTAPFCSWLQGFLGTLRCRFIAWFSS

SEQ ID NO: 29 - protein, 325 aa, Mus musculus
MKVFRRAWRHRVALGLGGLAFCGTTLLYLARCASEGETPSASGAARPRAKAFLAVLVASAPRAVERRTAVRSTWLAPERRGGPEDVWARFAVGTGGLGSEERRALELEQAQHGDLLLLPALRDAYENLTAKVLAMLTWVDERVDFEFVIKADDDSFARLDAILVDLRAREPARRRRLYWGFFSGRGRVKPGGRWREAAWQLCDYYLPYALGGGYVLSADLVHYIRLSREYLRAWHSEDVSLGTWLAPVDVQREHDPRFDTEYKSRGCNNQYLVTHKQSPEDMLEKQQMLLHEGRLCKHEVQLRLSYVYDWSAPPSQCCQRKEGVP